(19) United States
(12) Patent Application Publication
(10) Pub. No.: US 2019/0314470 A1
(43) Pub. Date: Oct. 17, 2019

(54) Methods Of Disrupting Ras-Driven Cancer Growth Using Engineered Bacterial Snare-Cleaving Toxins

(71) Applicants: United States Government As Represented By The Department of Veterans Affairs, Washington, DC (US); The Board of Trustees of The Leland Stanford Junior University, Stanford, CA (US)

(72) Inventors: Paul A. Khavari, Palo Alto, CA (US); Yonglu Che, Stanford, CA (US)

(21) Appl. No.: 16/149,766

(22) Filed: Oct. 2, 2018

Related U.S. Application Data
(60) Provisional application No. 62/566,757, filed on Oct. 2, 2017.

(51) Int. Cl. A61K 38/48; A61K 9/00; A61P 35/00

(52) U.S. Cl. CPC A61K 38/4893; A61K 45/06; A61P 35/00; A61K 9/0019

(57) ABSTRACT

Disclosed herein are methods of treating RAS-driven cancers using engineered bacterial SNARE-cleaving toxins.

### CROSS REFERENCE TO RELATED APPLICATIONS

This application claims the benefit of priority of U.S. Provisional Application No. 62/566,757 filed Oct. 2, 2017. The content of this earlier filed application is hereby incorporated by reference herein in its entirety.

### STATEMENT REGARDING FEDERALLY SPONSORED RESEARCH

This invention was made with Government support under contract AR043799 awarded by the National Institutes of Health. The Government has certain rights in the invention.

### INCORPORATION OF THE SEQUENCE LISTING

The present application contains a sequence listing that is submitted via EFS-Web concurrent with the filing of this application, containing the file name “37759_0160U1_SL.txt” which is 4,096 bytes in size, created on Oct. 1, 2018, and is herein incorporated by reference in its entirety.

### TECHNICAL FIELD OF THE INVENTION

The present invention relates generally to disruption of cancer growth and, in particular, to disruption of RAS-oncogene driven cancer growth using engineered bacterial SNARE-cleaving toxins.

### BACKGROUND

Mammalian cells express the closely related Ras proteins H-RAS, K-RAS, and N-RAS that, once mutated, become oncogenic.

The oncogene K-RAS, a member of the highly conserved RAS GTPase superfamily and a driver of human pancreatic, colon, and lung cancers (Stephen et al., Prior et al., Pylayeva-Gupta et al.), is an intricately regulated and difficult-to-target driver of human cancer.

Recently, K-RAS was shown to have RNA-binding capacity, directly binding sister non-coding RNAs SNORD50A and SNORD50B to suppress tumor formation (Siprashvili et al.). Further investigation reveals that SNORD50A/B compete with SNARE (soluble N-ethylmaleimide-sensitive factor attachment protein receptor) proteins for K-RAS binding. SNARE proteins are involved in membrane trafficking and exocytotic activity. In order to remain enriched on the plasma membrane where it can relay growth factor signaling, K-RAS undergoes cycles of endocytosis, solubilization, and vesicular transport back to the plasma membrane. The final step of this transport from recycling endosomes to the plasma membrane requires synaptosomal-associated proteins 23 (SNAP23) and 29 (SNAP29), and vesicle-associated membrane protein 3 (VAMP3) which are parts of a protein complex involved in the docking and fusion of synaptic vesicles. Competitive binding between SNAREs and SNORD50A/B to K-RAS moderate this process, and loss of SNORD50A/B leads to an increase in K-RAS signaling at the plasma membrane.

The lack of distinct allosteric pockets and the high affinity of GTP binding have made K-RAS a challenging therapeutic target despite its importance in cancer. Deeper understanding of the steps in the protein's lifecycle is important.

### SUMMARY

Disclosed herein are methods of disrupting RAS-driven cancer growth in a mammalian cell, said methods comprising administering to said cell an amount of a toxin derived from an engineered botulism toxin mutant effective in cleaving a SNARE protein that is essential for RAS signaling, thereby disrupting RAS-driven cancer growth.

Disclosed herein are methods of treating a patient affected with oncogenic Ras-driven cancer, the methods comprising administering to a patient a therapeutically effective amount of a botulism toxin (BoNT) isoform E mutant or biologically active variant thereof, wherein the BoNT isoform E cleaves SNAP23.

Disclosed herein are methods of reducing a tumor in a subject, wherein the tumor has a constitutively activating mutation of Ras, the methods comprising administering to the subject a therapeutically effective amount of botulism toxin (BoNT) isoform E mutant, wherein the BoNT isoform E mutant cleaves SNAP23.

Disclosed herein are methods of inhibiting or reducing K-RAS activity, the methods comprising contacting a cell or tissue with a therapeutically effective amount of an engineered bacterial SNARE-cleaving toxin.

Disclosed herein a methods of treating a subject with cancer, the methods comprising: (a) identifying a subject in need of treatment; and (b) administering to the subject a therapeutically effective amount of a toxin from an engineered Botulism toxin E mutant.

Disclosed herein are methods to disrupt recycling and sustained signaling of K-RAS using engineered bacterial SNARE-cleaving toxins based on the finding that competitive binding between SNORD50A/B snoRNAs and specific SNARE proteins control K-RAS localization and signaling.

Toxins from the engineered Botulism toxin E mutant (K224E) are shown to be capable of cleaving SNAP23 which is a SNARE protein that is important for tumorigenesis due to hyperactive RAS signaling. Thus, one embodiment herein is directed to a method of disrupting oncogenic RAS-driven cancer growth in a mammalian cell, said method comprising administering to said cell an amount of a toxin derived from an engineered botulism toxin mutant effective in cleaving a SNARE protein that is involved in RAS signaling, thereby disrupting RAS-driven cancer growth.

Other features and advantages of the present compositions and methods are illustrated in the description below, the drawings, and the claims.

### BRIEF DESCRIPTION OF THE DRAWINGS

FIGS. 1A-E show proteins whose proximity to K-RAS is affected by SNORD50A/B. FIG. 1A shows 197 K-RAS vicinal interactors, split into six quadrants, identified by mass spectrometry plotted using Stringdb. Edges represent either known protein-protein interactions or pathway interactions (3 WT cell lines, 6 SNORD50A/B KO clones). Characterization of SNORD50A/B KO clones can be found in (Siprashvili et al. 2015). FIG. 1A-1 shows the K-RAS vicinal interactors of quadrant 1. FIG. 1A-2 shows the K-RAS vicinal interactors of quadrant 2. FIG. 1A-3 shows the K-RAS vicinal interactors of quadrant 3. FIG. 1A-4 shows the K-RAS vicinal interactors of quadrant 4. FIG. 1A-5 shows the K-RAS vicinal interactors of quadrant 5. FIG. 1A-6 shows the K-RAS vicinal interactors of quadrant 6. FIG. 1B shows duplicate interaction networks from (A) highlighting the indicated annotations identified by Genemania (yellow) or by analysis (red). FIG. 1C shows K-RAS proximal proteins that are modulated by SNORD50A/B resolved by whether they are lost of gained upon SNORD50A/B knockout (SAINT probability score change of >0.25, replicable >1.5 fold change in 2 of 3 lines). FIG. 1D shows GO terms and KEGG pathway interactions associated with the SNORD50A/B-modulated interactome. FIG. 1E shows the abbreviated subset of the KEGG pathway: “SNARE interactions in vesicular transport”. K-RAS interactors identified in this disclosure are highlighted in red.

FIGS. 2A-L show that SNORD50A/B competitively inhibits SNARE proximity to K-RAS. FIG. 2A shows endogenous co-immunoprecipitation of K-RAS and SNAREs. FIG. 2B is a schematic of K-RAS mutants used in vicinal protein labeling experiments and strength of proximity labeling between Raf1, SNAP23, SNAP29, or VAMP3 with K-RAS mutants by proximity protein labeling followed by pulldown western blotting (n=2). FIG. 2C is the quantitation of (B). FIG. 2D shows PLA between K-RAS and either SNAP23, SNAP29, or VAMP3 as well as single antibody controls. Significance of WT cells determined by comparison to single antibody controls while significance of KO cells determined by comparison to WT. FIG. 2E shows the results of a far western blot with spotted purified recombinant protein indicated by row name. Bound K-RAS was detected with an anti-RAS antibody. Coomassie stain for total protein loading (middle) (n=3 representative images shown). FIG. 2F shows RNA competition in a far western of K-RAS interaction with SNAREs and canonical effector proteins; SCR=length and sequence nucleotide composition matched scrambled RNA control for SNORD50A/B (n=3, representative images shown). FIG. 2G is the quantitation of (F). Significance of SNAP23 and SNAP29 decrease calculated in comparidon to Raf1. FIGS. 2H-K shows the dissociation curves of K-RAS vs. SNORD50A (H), SNORD50B (I), SNAP23 (J), and SNAP29 (K). Kd values calculated from 3 independent experiments; U50=SNORD50A/B. FIG. 2L is a schematic showing SNORD50A/B and SNARE competitive binding.

FIGS. 3A-J show K-RAS recycling endosome-to-PM transport requires SNAREs. FIG. 3A shows the immunofluorescence of endogenous K-RAS in A549 cells. Side profile view of each cell (below). FIG. 3B shows K-RAS colocalization with the recycling endosome markers, Arf6 and Rab11. FIG. 3C and FIG. 3D show HRAS (C) and NRAS (D) colocalization with Arf6 and Rab11. FIGS. 3E and F shows CLIP-qPCR against SNORD50A (E) and SNORD50B (F) after immunoprecipitation of endogenous RAS in either cytoplasmic or membrane bound subcellular fractions (left). qPCR against SNORD50A and SNORD50B in total RNA isolated from cytoplasmic or membrane bound fractions (right). FIG. 3G show the proximity of K-RAS with membrane bound receptors and signaling effector proteins measured by PLA in H23; U50=SNORD50A/B. FIG. 3H show ERK and AKT activation in H23; quantitated replicate western blots are shown. FIG. 3I show a GSEA plot of K-RAS-dependent transcripts against RNA-sequencing datasets of SNARE wild-type and KO cells. FIG. 3J is a schematic of K-RAS recycling.

FIGS. 4A-I show that SNARE proteins are important for K-RAS-driven cancer. FIG. 4A shows the survival of K-RAS-mutant pancreatic adenocarcinoma patients depending on mutation and expression status of SNAP23 and VAMP3 (n=186). FIG. 4B show the survival of K-RAS-mutant pancreatic adenocarcinoma patients depending on mutation and expression status of the 36 other SNARE proteins (n=162). FIG. 4C shows subcutaneous in vivo tumor growth of H23 SHO immune deficient mice (n=4 per condition). FIG. 4D show subcutaneous in vivo tumor growth in DLD-1 isogenic lines (n=4 per condition). FIG. 4E is a schematic of a focused CRISPR screen targeting K-RAS, HRAS, NRAS, SNAP23, SNAP29, and VAMP3 (n=9 tumors). FIG. 4F shows the correlation of in vivo tumorigenesis sgRNA counts by target gene in CRISPR screen. FIG. 4G is a schematic of WT BoNT light chain E(LC/E) and LC/E-K224E bound to SNAP25 and SNAP23. FIG. 4H shows the growth in culture of H23 measured by CTB (n=2). FIG. 4I shows the tumor volumes of H23 at day 16 (n=10 per condition).

FIGS. 5A-D show KRAS proximal proteins by proximal protein labeling. FIG. 5A is a schematic of proximity protein labeling experiments. FIG. 5B shows a workflow for BioID-mass spectrometry and SAINT scoring. FIG. 5C shows spectral counts by protein (row) and sample (column) from mass spectrometry analysis of K-RAS vicinal proteins. FIG. 5D shows Gene Ontology (GO) terms enriched in WT (FIG. 5D-1) and SNORD50A/B KO (5D-2) cell lines.

FIGS. 6 A-H show SNARE and SNORD50A/B interactions with KRAS. FIG. 6A shows PLA between K-RAS and either SNAP23, SNAP29, or VAMP3 as well as single antibody controls. PLA signal in red, nuclei in blue. FIG. 6B shows the quantification of anti-RAS blot from FIG. 2E. FIGS. 6C-H shows the microscale thermophoresis response curves of K-RAS vs. SNORD50A (C), (D) SNORD50B (E) SNAP23+SCR RNA (F) SNAP23+SNORD50A/B (G) SNAP29+SCR RNA (H) SNAP29+SNORD50A/B.

FIGS. 7A-C show CRISPR-Cas9 mediated SNARE knockouts. FIG. 7A shows the representative western blots and diagram of CRISPR mediated SNARE KO. FIG. 7B shows the quantitation of KO efficiency. FIG. 7C shows the representative western blots and schematic of bulk population SNARE triple KOs.

FIGS. 8A-D show that SNARE knockouts disrupts KRAS signaling and target gene expression. FIG. 8A shows representative PLA images of RASGTP interaction with EGFR, P110α, and Raf1 in A549. F-actin labeled in green. FIG. 8B shows the quantification of the PLA signal in (A). FIG. 8C shows immunoblots of active/total AKT and ERK. FIG. 8D shows gene sets lost upon SNARE TKO in H23, A549, and CHL1 from RNA sequencing.

FIGS. 9A-F show mutation, downregulation, and expression of SNAREs in human cancer. FIG. 9A shows SNAP23 and VAMP3 expression by RNAseq in K-RAS WT and MUT TCGA patients. FIG. 9B shows mRNA expression of SNAP23 and VAMP3 expression patients with pancreatic adenocarcinoma. FIG. 9C shows colorectal adenocarcinoma percent survival. FIG. 9D shows the percent survival of SNAP23/VAMP3 mutation or downregulation to unaltered. FIG. 9E shows the survival ratio of SNAP23/VAMP3 mutation or downregulation to unaltered. FIG. 9F shows PLA of RAS-SNARE contact in human colorectal cancer tissue. FIG. 9G shows quantified PLA in 9 primary colorectal cancer tissue samples.

FIGS. 10A-H show SNARE knockouts disrupt KRAS-driven tumorigenesis. FIG. 10A shows representative tumor images from H23 tumorigenesis (tumors too small to be excised were omitted). FIG. 10B shows the final extracted tumors from DLD-1 tumorigenesis. FIG. 10C-D shows anchorage-independent growth (C) and growth in culture of isogenic DLD-1 cells (D). FIG. 10E shows sgRNA fold change between final tumor and initial cell population by cell line. FIG. 10F shows the fold change of target genes in selected cell lines. FIG. 10G shows L32 qPCR loading control of BoNT expressing H23s (H) qPCR of BoNT expression in H23s for FIGS. 4H and (I).

FIGS. 11A-D show mass spectometry spectral counts.

FIG. 12 shows human SNARE proteins.

FIG. 13 CRISPR screen guide counts.

FIG. 14 shows TCGA survival.

FIG. 15 shows sgRNA target sequences.

### DETAILED DESCRIPTION

The present disclosure can be understood more readily by reference to the following detailed description of the invention, the figures and the examples included herein.

Before the present compositions and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, the term “subject” refers to the target of administration, e.g., a human. Thus the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment for cancer, such as, for example, prior to the administering step.

As used herein, the term “contacting” refers to bringing a disclosed composition, compound, conjugate or fusion protein together with an intended target (such as, e.g., a cell or population of cells, a receptor, an antigen, or other biological entity) in such a manner that the disclosed composition, compound, conjugate or fusion protein can affect the activity of the intended target (e.g., receptor, transcription factor, cell, population of cells, etc.), either directly (i.e., by interacting with the target itself), or indirectly (i.e., by interacting with another molecule, co-factor, factor, or protein on which the activity of the target is dependent). In an aspect, a disclosed composition or fusion protein can be contacted with a cell or population of cells, such as, for example, one or more lymphocytes (e.g., T cells and/or B cells).

As used herein, the term “inhibit” or “inhibiting” or “reducing” mean decreasing tumor cell growth rate from the rate that would occur without treatment and/or causing tumor mass (e.g., cancer) to decrease. Inhibiting or reducing also include causing a complete regression of the tumor (e.g., cancer).

As used herein, the term “derived from” is used to identify the original source of a molecule but is not meant to limit the method by which the molecule is made which can be made, for example, by chemical synthesis or recombinant means.

As used herein, the terms “variant” and “mutant” refer to biologically active derivatives of the reference molecule, that retain desired activity. For example, an engineered botulism toxin mutant can have the same, altered or improved ability to cleave one or more SNARE proteins. In general, the term “variant” in reference to a polypeptide refer to compounds having a native polypeptide sequence and structure with one or more amino acid additions, substitutions (generally conservative in nature) and/or deletions, relative to the native molecule, so long as the modifications do not destroy biological activity and which are “substantially homologous” to the reference molecule as defined below. In general, the amino acid sequences of such variants will have a high degree of sequence homology to the reference sequence, e.g., amino acid sequence homology of more than 50%, generally more than 60%-70%, even more particularly 80%-85% or more, such as at least 90%-95% or more, when the two sequences are aligned. Often, the variants will include the same number of amino acids but will include substitutions. The term “mutant” refers to a protein that can have a single amino acid change or wide-range amino acid changes.

As explained above, variants generally include substitutions that are conservative in nature, i.e., those substitutions that take place within a family of amino acids that are related in their side chains. Specifically, amino acids are generally divided into four families: (1) acidic-aspartate and glutamate; (2) basic-lysine, arginine, histidine; (3) non-polar-alanine, valine, leucine, isoleucine, proline, phenylalanine, methionine, tryptophan; and (4) uncharged polar-glycine, asparagine, glutamine, cysteine, serine threonine, tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the polypeptide of interest may include up to about 5-10 conservative or non-conservative amino acid substitutions, or even up to about 15-25 conservative or non-conservative amino acid substitutions, or any integer between 5-25, so long as the desired function of the molecule remains intact. One of skill in the art may readily determine regions of the molecule of interest that can tolerate change.

Disclosed herein are results from in vivo tumorigenesis and analysis of The Cancer Genome Atlas (TCGA) pancreatic cancer revealing co-importance between specific SNAREs and K-RAS, and in particular, important functions of SNAP23 and VAMP3 in K-RAS-driven cancers.

Analysis of 5,473 tumor-normal genome pairs revealed SNORD50A and SNORD50B deletion in 10-40% of 12 human cancers and several cases where their deletion was associated with shortened survival. SNORD50A/B are named and annotated as C/D box small nucleolar RNAs that canonically associate with Fibrillarin, Nop56p, Snu13p, and Nop58p to form a ribonucleoprotein complex where the snoRNA directs 2′-O-methylation of complimentary ribosomal RNA (Williams et al.). SNORD50A/B were reported to bind K-RAS as detected by electromobility shift assay and crosslinking followed by immunoprecipitation (CLIP) (Jensen et al.), and genomic deletion of SNORD50A/B led to hyperactive Ras-ERK signaling and accelerated tumorigenesis (Siprashvili et al.). This suggests that snoRNAs may have protein-binding functions outside of their canonical ribonucleoprotein complexes, and in particular, SNORD50A/B may have dampening roles on K-RAS signaling.

Analysis of SNORD50A/B deletions and oncogenic K-RAS mutations in cancer revealed highly co-occurrent alterations of SNORD50A/B and K-RAS, suggesting that unlike mutually-exclusive Ras/Raf mutations, SNORD50A/B regulate K-RAS through a mechanism orthogonal to direct signal transduction. SNARE proteins (Soluble NSF Attachment Protein Receptor) are large protein complexes that are involved in mediating the fusion of vesicles with their target membrane compartments. SNAP23 is an example of a SNARE. Some SNAREs are targets of bacterial neurotoxins.

Botulinum Toxin (BoNT), derived from the Clostridium sp strains, is a proteolytic enzyme that cleaves SNARE proteins in neurons. Eight isotypes of BoNT are known, and each isotype has a different cleavage site on SNARE proteins. SNAP23 is resistant to cleavage by by BoNT isotype E. Cleavage of SNAP23 by a BoNT isotype E mutant can inhibit the function of the SNAP23 in forming a SNARE complex for fusion of vesicles to membrane. Engineered, modified or variant BoNT with specificity for non-neuronal SNAREs are within the scope of this disclosure.

Ras proteins are GTPase proteins involved in transmitting signals within cells. When Ras is ‘switched on’ by an incoming signal, it then can switch on other proteins that can turn on genes that are involved in cell growth, differentiation and survival. Mutations in ras genes can lead to constitutively active or permanently activated Ras proteins. Overactive signaling inside the cell can lead to or has been associated with cancer. Examples of Ras oncogenes in human cancer include HRas, KRas and NRas.

Methods of Treatment

Disclosed herein are methods of treating a patient affected with oncogenic Ras-driven cancer. Also disclosed herein are methods of reducing a tumor in a subject. Also disclosed herein, are methods of treating a patient with cancer. In an aspect, the tumor has a constitutively activating mutation of Ras. In some aspects, the method can comprise administering to the patient or subject a therapeutically effective amount of a botulism toxin (BoNT) isoform E mutant. In some aspects, the method can comprising identifying the patient in need of treatment. In some aspects, the botulism toxin isoform E mutant can be engineered or a biologically active variant thereof. In some aspects, the BoNT isoform E mutant or biologically active variant thereof cleaves SNAP23. In an aspect, the botulism toxin isoform E mutant can comprise a K224E mutation. In an aspect, the SNARE protein can be SNAP23.

Disclosed herein are methods of inhibiting or reducing K-RAS activity. Also disclosed herein are methods of inhibiting or reducing K-RAS signaling. In an aspect, the methods can comprise contacting a cell or tissue with a therapeutically effective amount of an engineered bacterial SNARE-cleaving toxin. In an aspect, the SNARE-cleaving toxin can be a toxin from an engineered Botulism toxin E mutant. In an aspect, the engineered Botulism toxin E mutant can comprise a K224E mutation. In an aspect, the SNARE protein can be SNAP23.

Also disclosed herein are methods of disrupting RAS-driven cancer growth in a mammalian cell. In an aspect, the method can comprise administering to said cell an amount of a toxin derived from an engineered botulism toxin mutant effective in cleaving a SNARE protein. In an aspect, the SNARE protein can be essential for RAS signaling. In an aspect, the method can disrupt RAS-driven cancer growth. By “disrupt”, the RAS-driven cancer growth can be reduced or inhibited or slowed compared to a cell that has not be administered a toxin derived from an engineered botulism toxin mutant effective in cleaving a SNARE protein. In an aspect, the SNARE protein can be SNAP23.

The BoNT isoform E mutant or biologically active variant or the engineered Botulism toxin E mutant can be formulated to include a therapeutically effective amount. Therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to a type of cancer or a RAS-driven cancer.

The compositions described herein can be administered to the subject (e.g., a human patient) in an amount sufficient to delay, reduce, or preferably prevent the onset of clinical disease. Accordingly, in some aspects, subject can be a human. In some aspects, the patient can be a human patient. In therapeutic applications, compositions can be administered to a subject (e.g., a human patient) already with or diagnosed with cancer in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a composition (e.g., a pharmaceutical composition) can be an amount that achieves a cure, but that outcome is only one among several that can be achieved. As noted, a therapeutically effective amount includes amounts that provide a treatment in which the onset or progression of the cancer is delayed, hindered, or prevented, or the cancer or a symptom of the cancer is ameliorated. One or more of the symptoms can be less severe. Recovery can be accelerated in an individual who has been treated.

Disclosed herein, are methods of treating a patient with cancer. The cancer can be any cancer. In some aspects, the cancer can be a primary or secondary tumor. In other aspects, the primary or secondary tumor can within the patient's pancreas, lung, colon, rectum, thyroid, bladder, liver, kidney, skin, cervix, head or neck. In some aspects, the cancer can be lung cancer, colon cancer, pancreatic cancer, colorectal cancer, cervical cancer, head and neck squamous cell carcinoma, thyroid cancer, melanoma, bladder cancer, liver cancer, or kidney cancer. In an aspect, the subject has been diagnosed with cancer prior to the administering step. In an aspect, the cancer can be a RAS-driven cancer. In an aspect, the cancer can have or be associated with a constitutively activating mutation of RAS. In an aspect, the patient can have a tumor that can be a RAS-driven tumor. In an aspect, the tumor can have or be associated with a constitutively activating mutation of RAS.

The compositions described herein can be formulated to include a therapeutically effective amount of botulism toxin (BoNT) isoform E mutant. In an aspect, the botulism toxin (BoNT) isoform E mutant can be contained within a pharmaceutical formulation. In an aspect, the pharmaceutical formulation can be a unit dosage formulation.

The therapeutically effective amount or dosage of the botulism toxin (BoNT) isoform E mutant used in the methods as disclosed herein applied to mammals (e.g., humans) can be determined by one of ordinary skill in the art with consideration of individual differences in age, weight, sex, other drugs administered and the judgment of the attending clinician. Variations in the needed dosage may be expected. Variations in dosage levels can be adjusted using standard empirical routes for optimization. The particular dosage of a pharmaceutical composition to be administered to the patient will depend on a variety of considerations (e.g., the severity of the cancer symptoms), the age and physical characteristics of the subject and other considerations known to those of ordinary skill in the art. Dosages can be established using clinical approaches known to one of ordinary skill in the art.

The duration of treatment with any composition provided herein can be any length of time from as short as one day to as long as the life span of the host (e.g., many years). For example, the compositions can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compositions can be administered once (or twice, three times, etc.) daily, weekly, monthly, or yearly.

The total effective amount of the compositions as disclosed herein can be administered to a subject as a single dose, either as a bolus or by infusion over a relatively short period of time, or can be administered using a fractionated treatment protocol in which multiple doses are administered over a more prolonged period of time. Alternatively, continuous intravenous infusions sufficient to maintain therapeutically effective concentrations in the blood are also within the scope of the present disclosure.

The compositions described herein can be administered in conjunction with other therapeutic modalities to a subject in need of therapy. The present compounds can be given to prior to, simultaneously with or after treatment with other agents or regimes. For example, botulism toxin (BoNT) isoform E mutant disclosed herein can be administered in conjunction with standard therapies used to treat cancer. In an aspect, compositions described herein can be administered or used together with chemotherapy. In some aspects, the BoNT isoform E mutant can be administered in combination with one or more chemotherapeutic or anti-cancer therapeutics. In some aspects, BoNT isoform E mutant can be administered in combination with radiotherapy or immunotherapy.

Pharmaceutical Compositions

As disclosed herein, are pharmaceutical compositions, comprising botulism toxin (BoNT) isoform E mutant or a biologically variant thereof as disclosed herein. As disclosed herein, are pharmaceutical compositions, comprising botulism toxin (BoNT) isoform E mutant and a pharmaceutical acceptable carrier described herein. In some aspects, botulism toxin (BoNT) isoform E mutant can be formulated for oral or parental administration. In some aspects, botulism toxin (BoNT) isoform E mutant can be administered parentally or intravenously. In an aspect, the parental administration can be intravenous, subcutaneous, intramuscular or direct injection. The compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

The compositions can be administered directly to a subject. Generally, the compositions can be suspended in a pharmaceutically acceptable carrier (e.g., physiological saline or a buffered saline solution) to facilitate their delivery. Encapsulation of the compositions in a suitable delivery vehicle (e.g., polymeric microparticles or implantable devices) may increase the efficiency of delivery.

The compositions can be formulated in various ways for parenteral or nonparenteral administration. Where suitable, oral formulations can take the form of tablets, pills, capsules, or powders, which may be enterically coated or otherwise protected. Sustained release formulations, suspensions, elixirs, aerosols, and the like can also be used.

Pharmaceutically acceptable carriers and excipients can be incorporated (e.g., water, saline, aqueous dextrose, and glycols, oils (including those of petroleum, animal, vegetable or synthetic origin), starch, cellulose, talc, glucose, lactose, sucrose, gelatin, malt, rice, flour, chalk, silica gel, magnesium stearate, sodium stearate, glycerol monosterate, sodium chloride, dried skim milk, glycerol, propylene glycol, ethanol, and the like). The compositions may be subjected to conventional pharmaceutical expedients such as sterilization and may contain conventional pharmaceutical additives such as preservatives, stabilizing agents, wetting or emulsifying agents, salts for adjusting osmotic pressure, buffers, and the like. Suitable pharmaceutical carriers and their formulations are described in “Remington's Pharmaceutical Sciences” by E. W. Martin, which is herein incorporated by reference. Such compositions will, in any event, contain an effective amount of the compositions together with a suitable amount of carrier so as to prepare the proper dosage form for proper administration to the patient.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used. Thus, compositions can be prepared for parenteral administration that includes botulism toxin (BoNT) isoform E mutant dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like).

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules.

In an aspect, a pharmaceutical composition comprises botulism toxin (BoNT) isoform E mutant and optionally, a pharmaceutical acceptable carrier. Further, the pharmaceutical composition comprises botulism toxin (BoNT) isoform E mutant in therapeutically effective amounts.

Articles of Manufacture

The composition described herein can be packaged in a suitable container labeled, for example, for use as a therapy to treat cancer, method of reducing a tumor in a subject, wherein the tumor has a constitutively activating mutation of RAS, treating a patient affected with oncogenic RAS-driven cancer, inhibiting or reducing K-RAS signaling or activity or any of the methods disclosed herein. Accordingly, packaged products (e.g., sterile containers containing the composition described herein and packaged for storage, shipment, or sale at concentrated or ready-to-use concentrations) and kits, including at least imipramine as described herein and instructions for use, are also within the scope of the disclosure. A product can include a container (e.g., a vial, jar, bottle, bag, or the like) containing the composition described herein. In addition, an article of manufacture further may include, for example, packaging materials, instructions for use, syringes, buffers or other control reagents for treating or monitoring the condition for which prophylaxis or treatment is required. The product may also include a legend (e.g., a printed label or insert or other medium describing the product's use (e.g., an audio- or videotape)). The legend can be associated with the container (e.g., affixed to the container) and can describe the manner in which the compound therein should be administered (e.g., the frequency and route of administration), indications therefor, and other uses. The compounds can be ready for administration (e.g., present in dose-appropriate units), and may include a pharmaceutically acceptable adjuvant, carrier or other diluent. Alternatively, the compounds can be provided in a concentrated form with a diluent and instructions for dilution.

### EXAMPLES

### Example 1: SNORD50A- and SNORDB-K-RAS

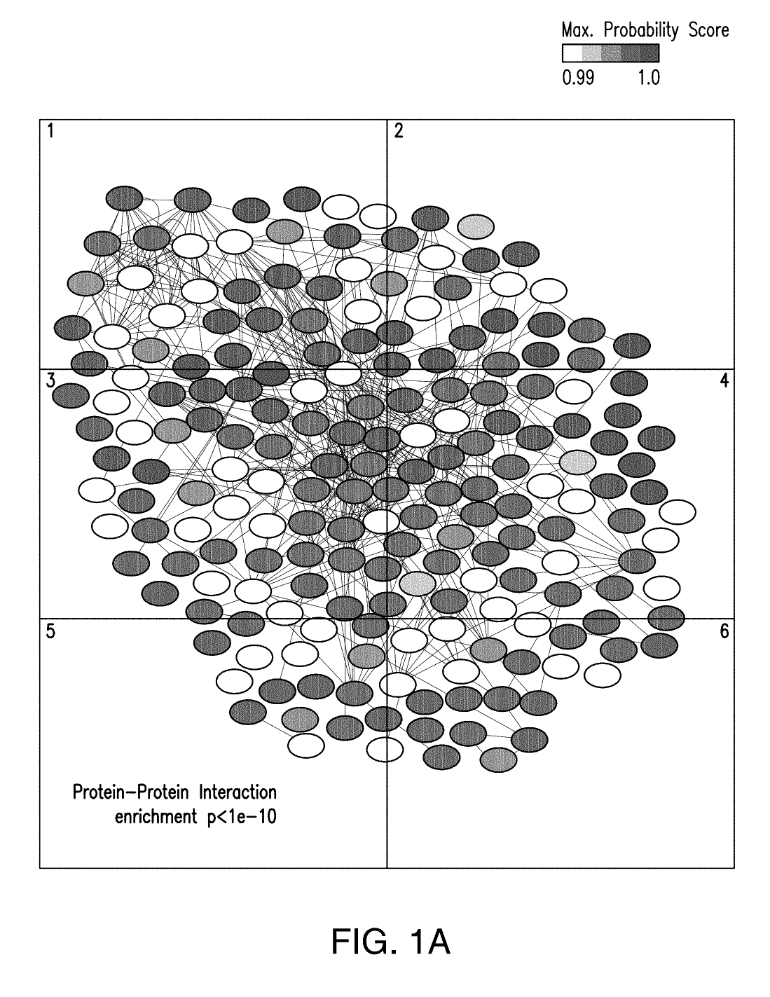

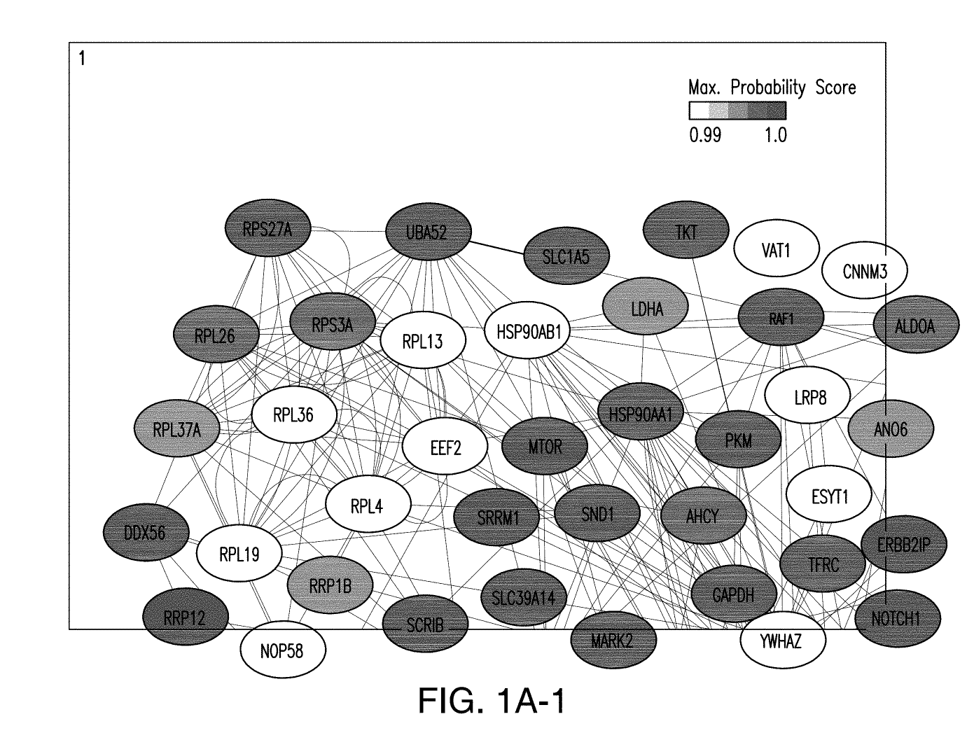

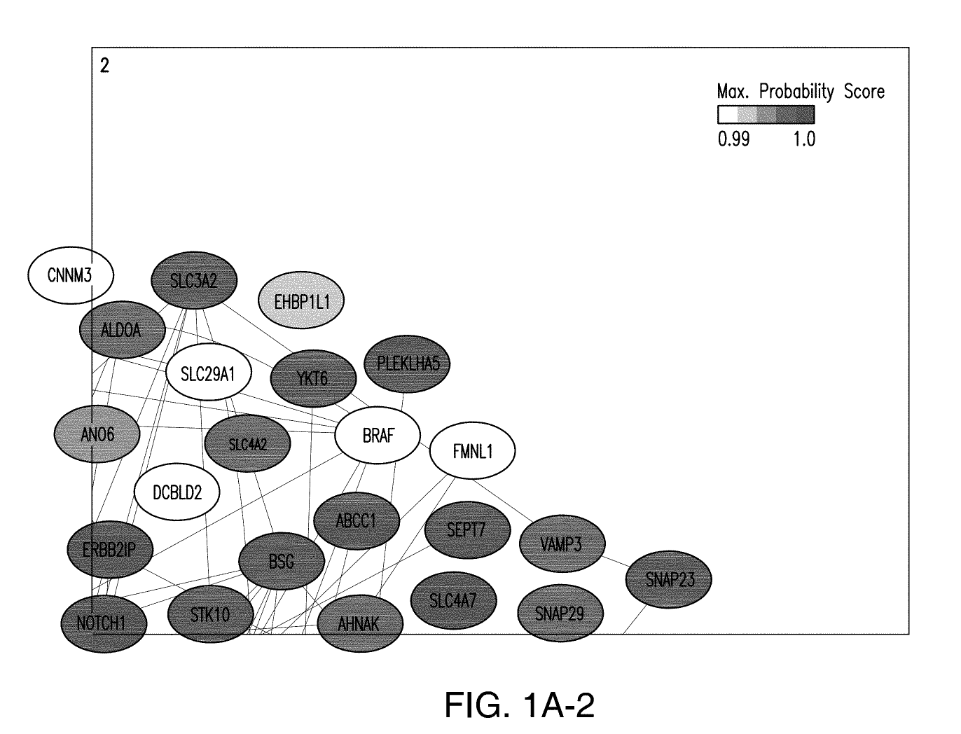

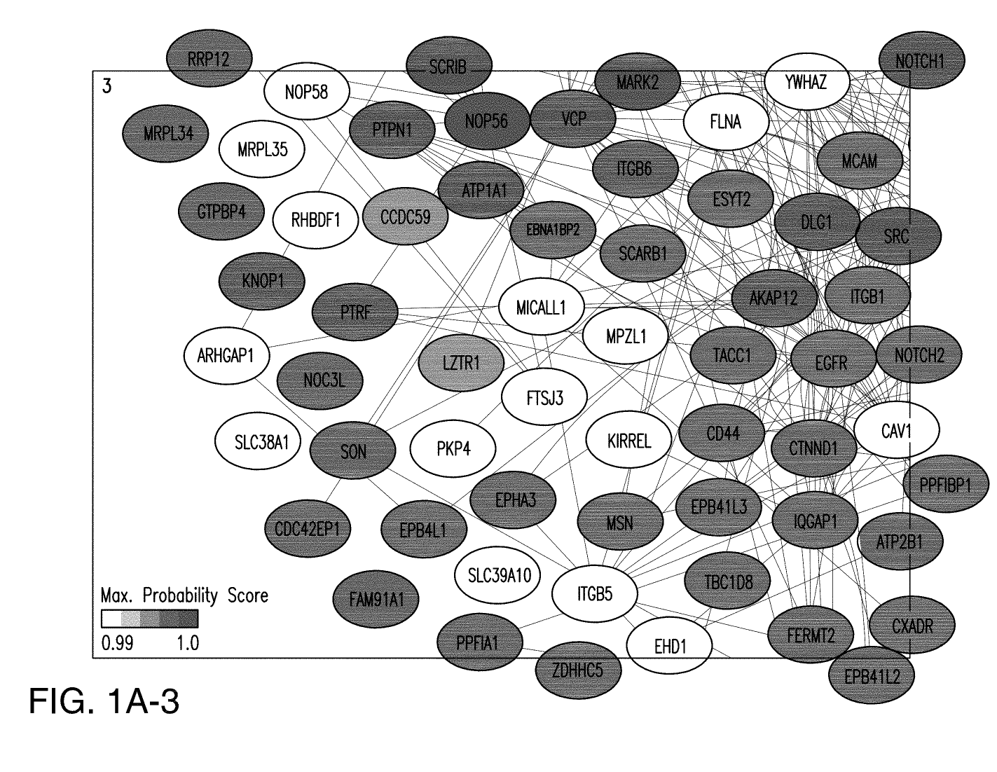

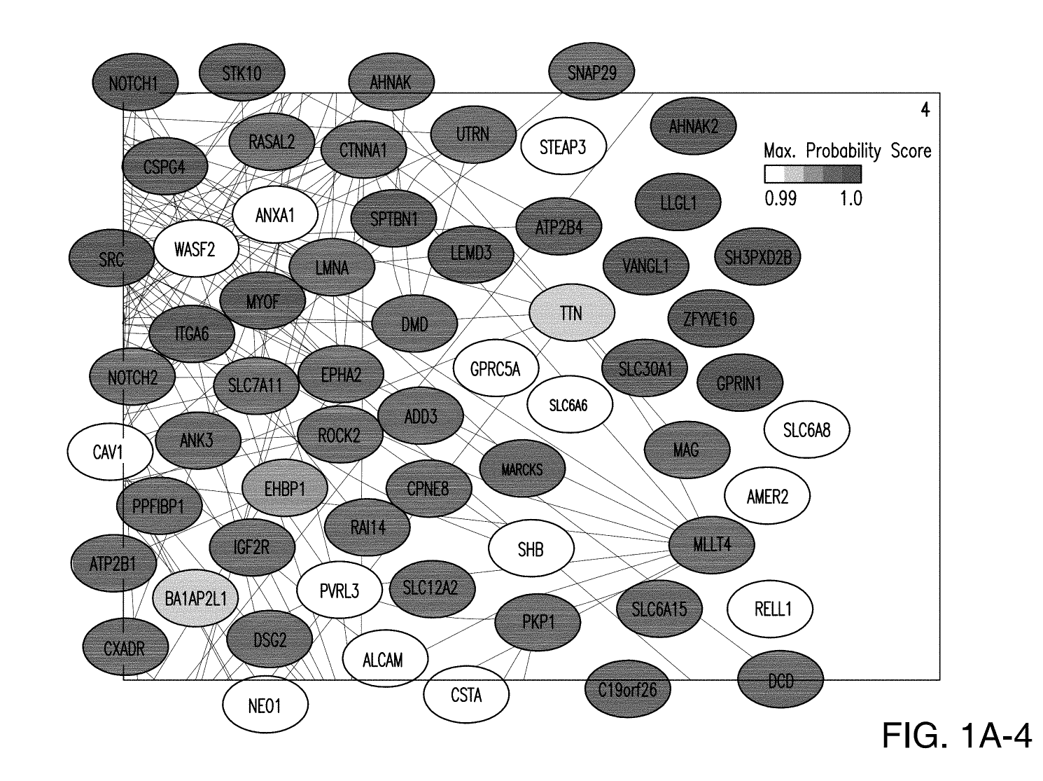

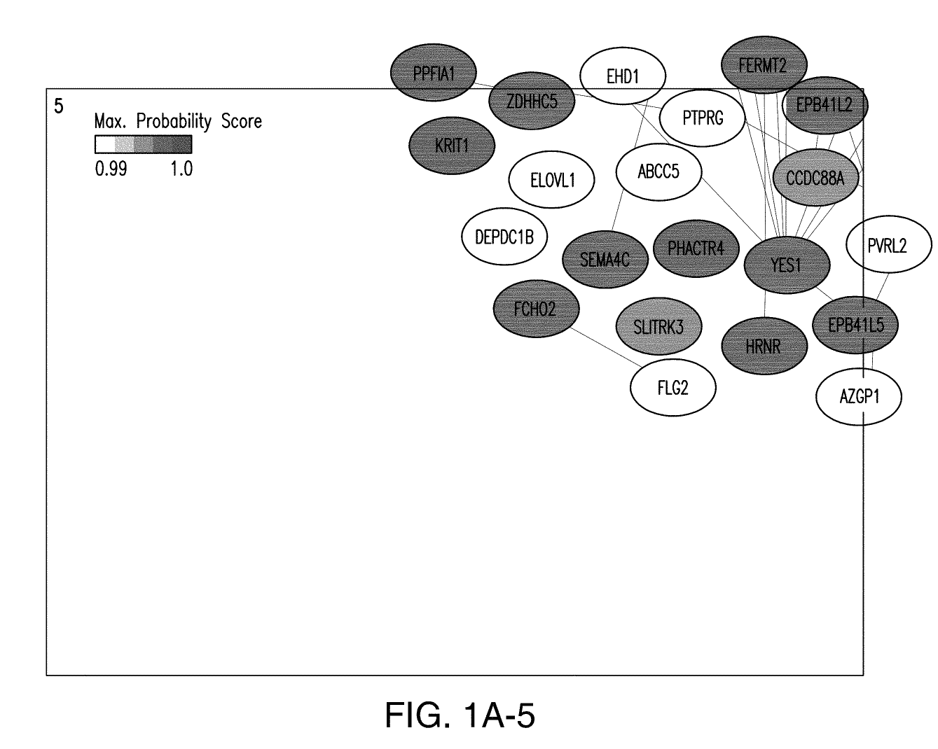

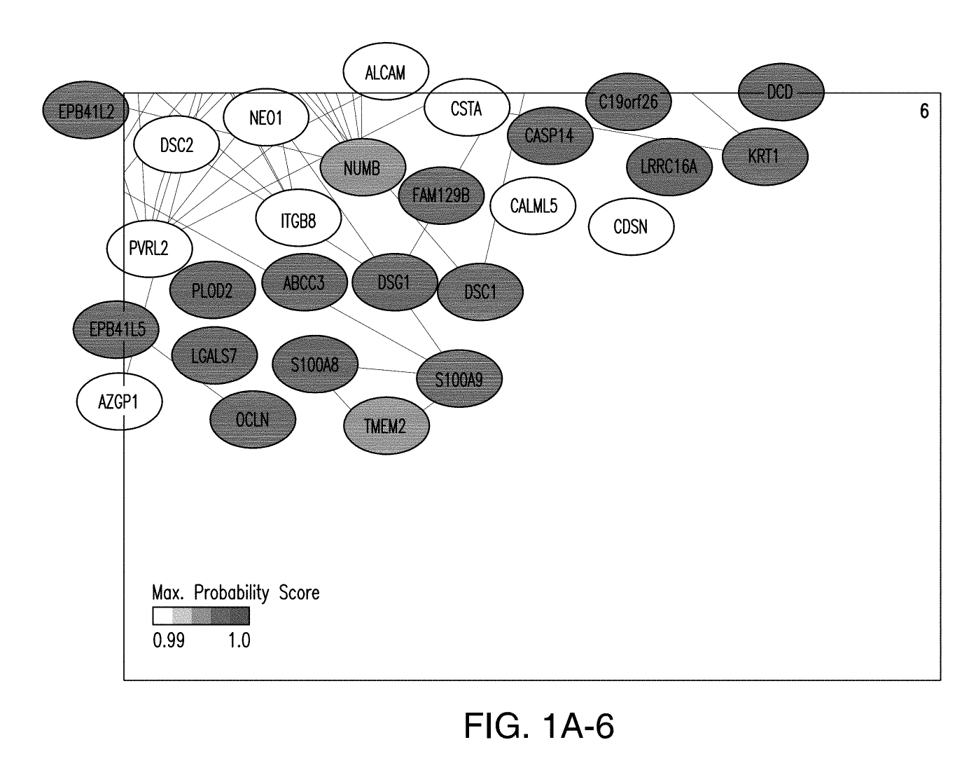

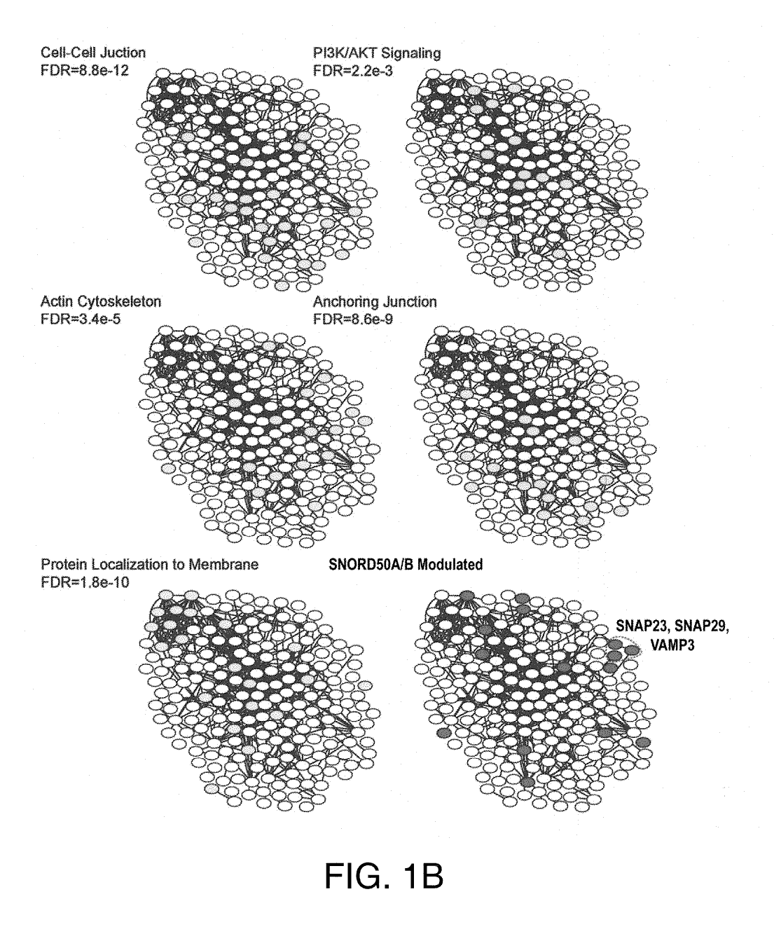

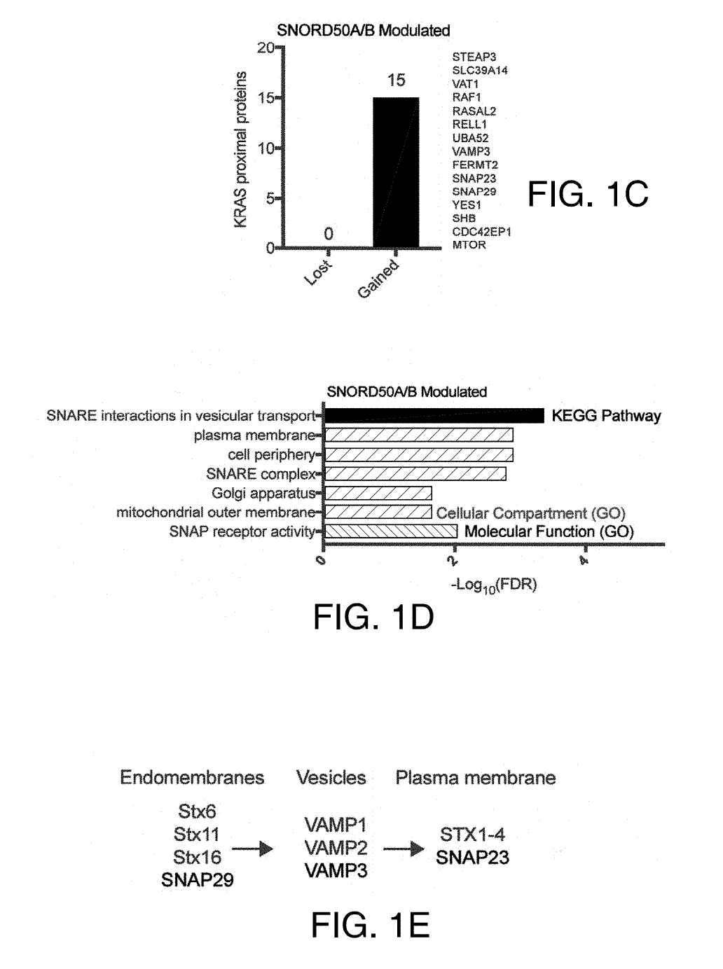

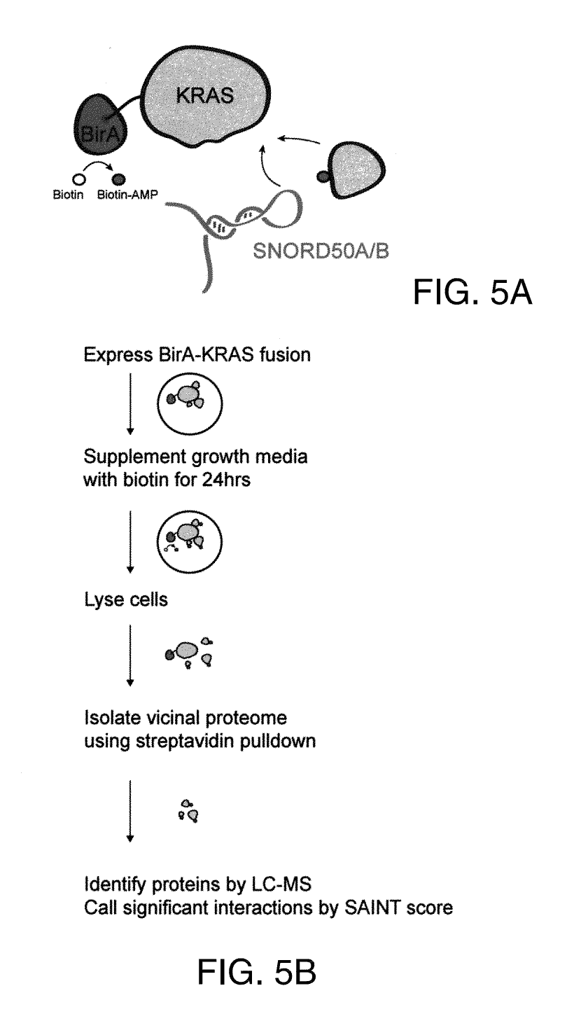

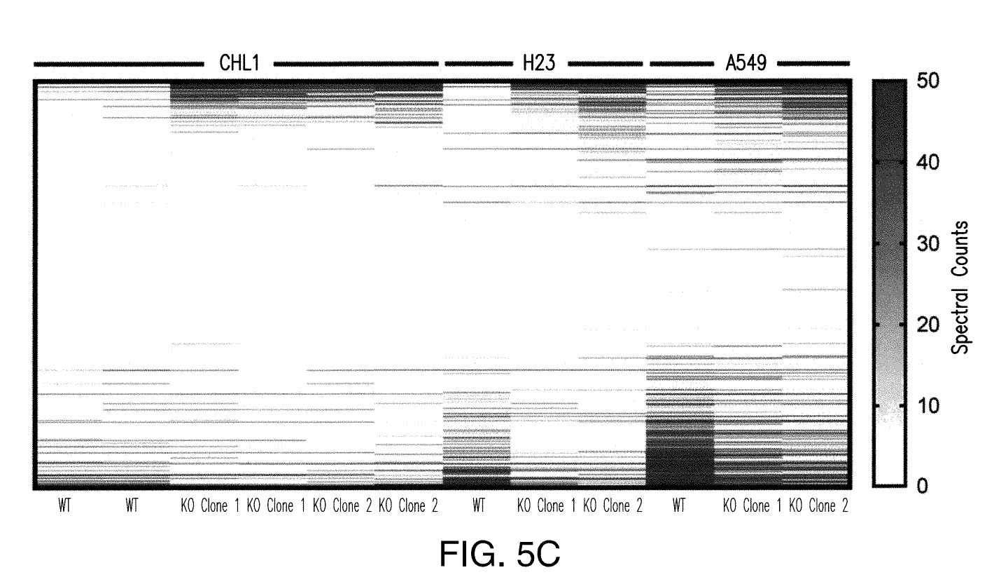

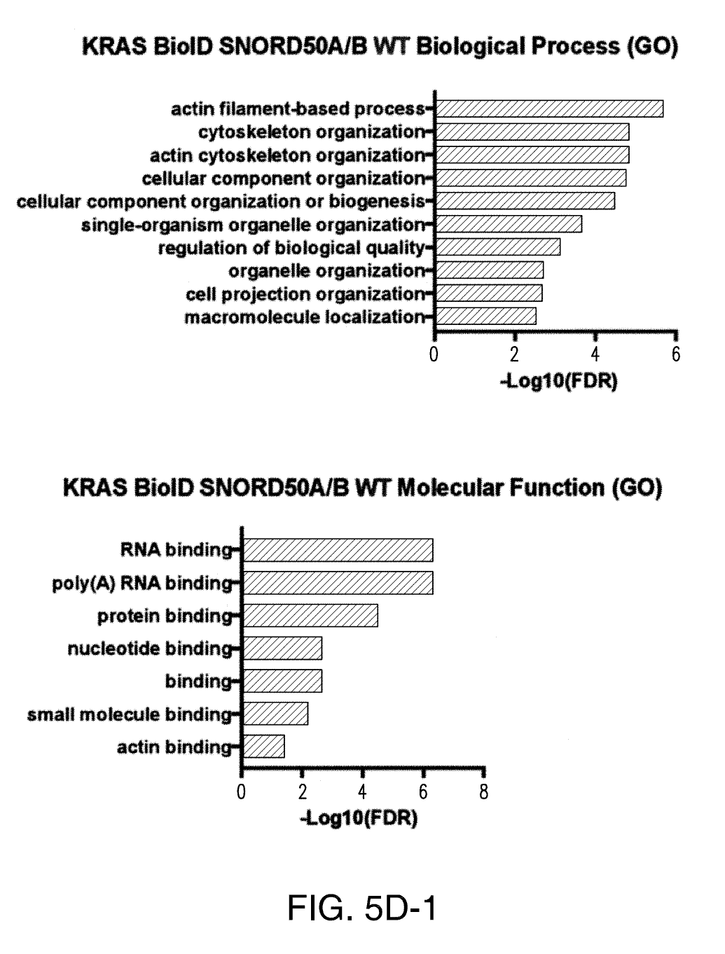

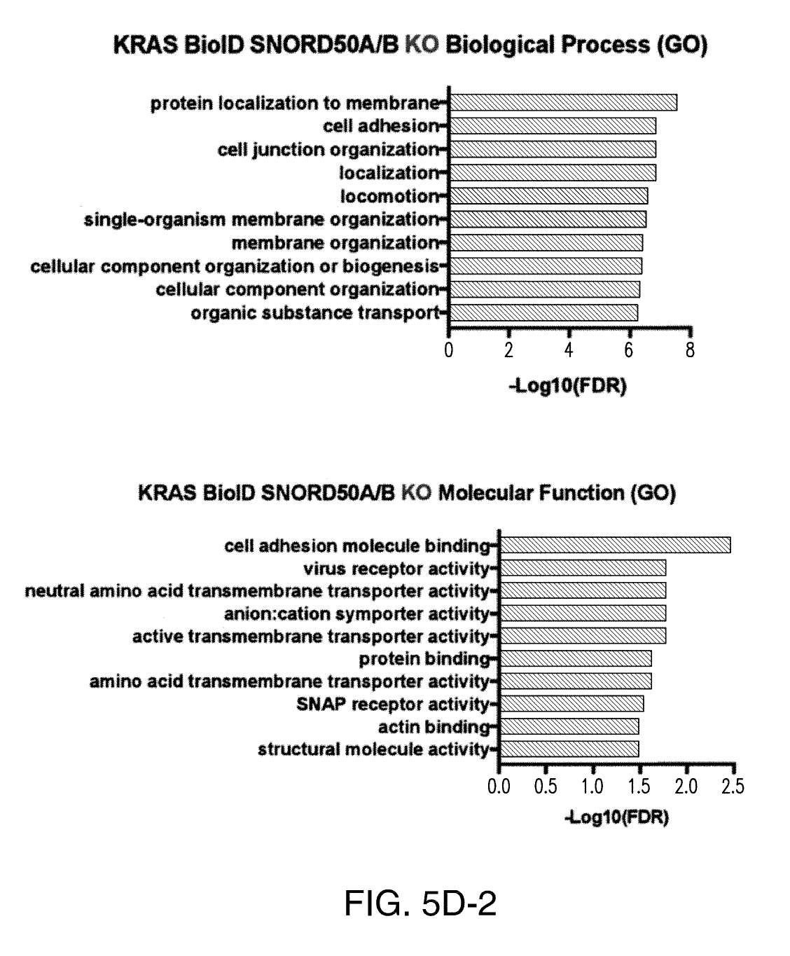

To broadly screen how K-RAS function is altered by SNORD50A/B binding, a combined genetic and proteomics approach was used to study the proximal protein interactome of K-RAS and the changes incurred upon SNORD50A/B loss. Duplicate SNORD50A/B-deleted subclones were generated from three independent human cancer cell lines (H23, A549, CHL1). In each of the nine total cell lines (3 WT, 6 SNORD50A/B KO), K-RAS was expressed with an N-terminal BirA fusion on a flexible linker. BirA is a promiscuous biotin ligase that releases reactive biotin intermediates with a spatially limited half-life of 10-20 nm (Roux et al.). Biotin stimulation of BirA-K-RAS labels spatially-proximal protein interactions that can be later captured by LC/MS mass spectometry (FIG. 5a, b). Proximal protein labeling of K-RAS reveals a highly-interconnected interactome enriched in annotations consistent with Ras biology including membrane localization and PI3K/AKT signaling (FIGS. 1a,b FIG. 5c,d) (Szklarczyk et al.).

The difference between SNORD50A/B WT and KO lines was assessed by SAINT probability score of pooled datasets from respective samples. Fifteen proteins differed in SAINT score by >0.25 with replicable fold change differences in spectral counts in at least 2 of 3 cell lines suggesting that SNORD50A/B may affect the interaction of these 15 proteins with K-RAS (FIG. 1c). Among the 15 candidate SNORD50A/B-modulated interactors, 15 were enriched in SNORD50A/B KO over WT cells. This one-sided enrichment of SNORD50A/B-modulated interactors suggests competitive action between SNORD50A/B and protein interactors of K-RAS.

Further analysis of the functional annotations of these 15 proteins reveals enrichment of a single significant KEGG pathway: SNARE interactions in vesicular transport (FIG. 1d). The SNARE proteins SNAP29, VAMP3, and SNAP23 identified in the SNORD50A/B-modulated proximal interactome are suggested to mediate endosome-to-plasma membrane traffic (FIG. 1e). This unifying pathway and strong modulation by SNORD50A/B led to the further characterization of these SNAREs.

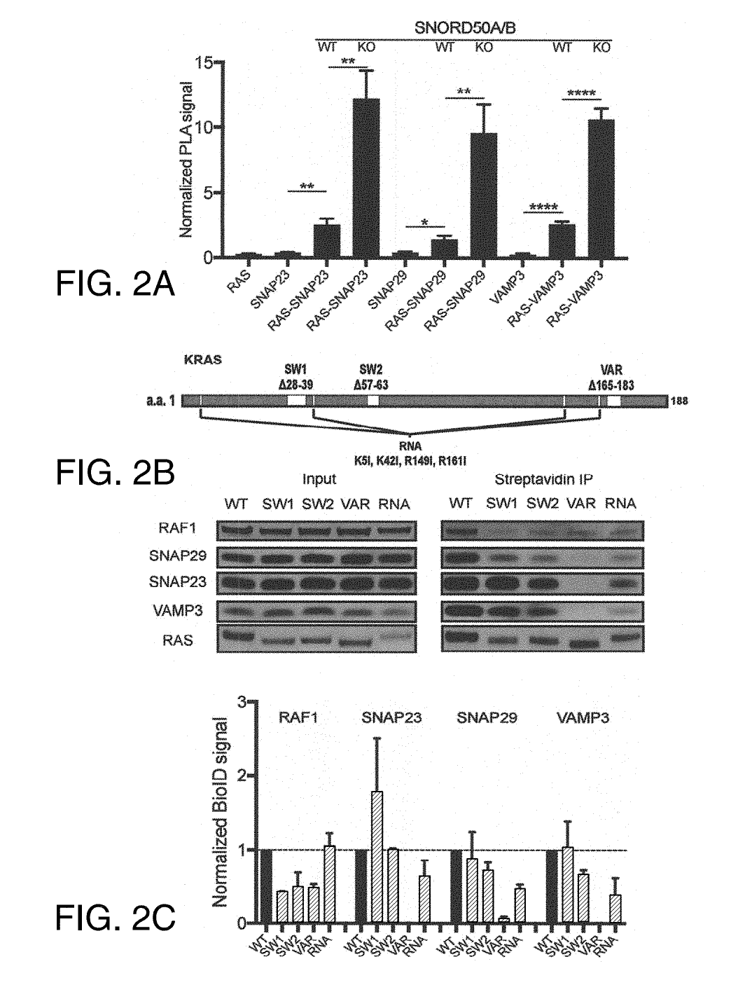

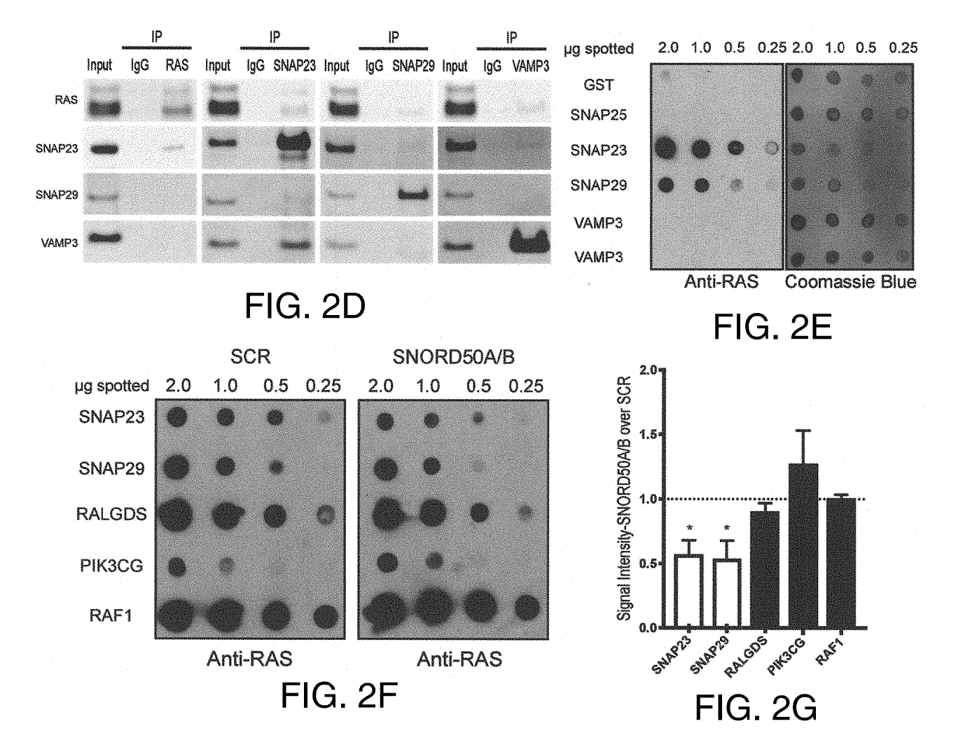

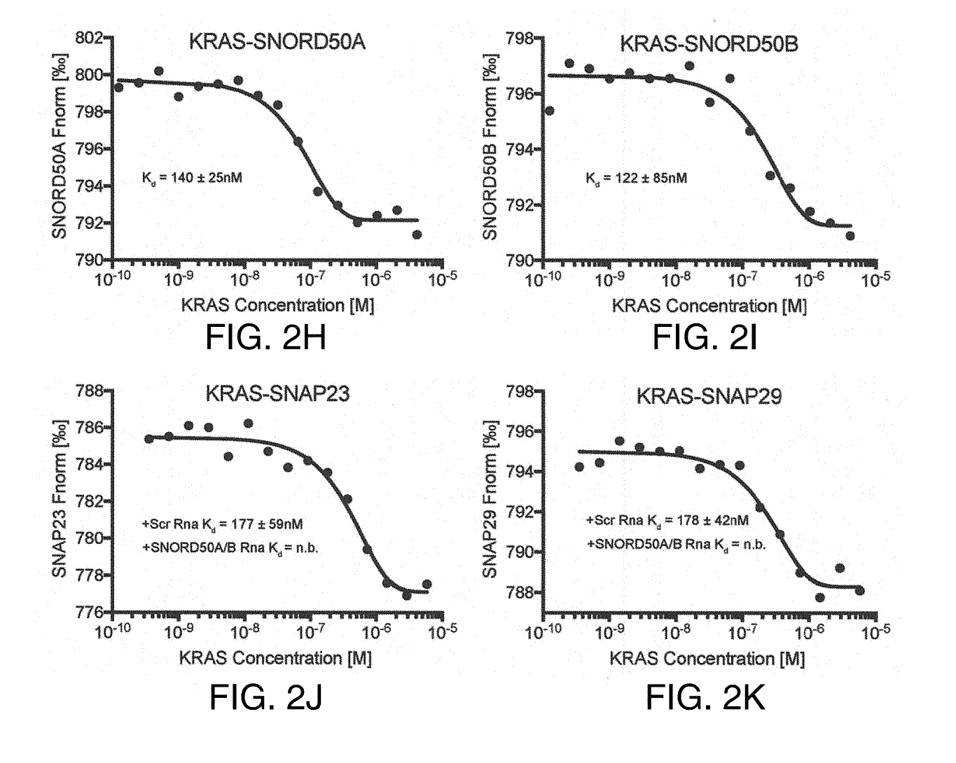

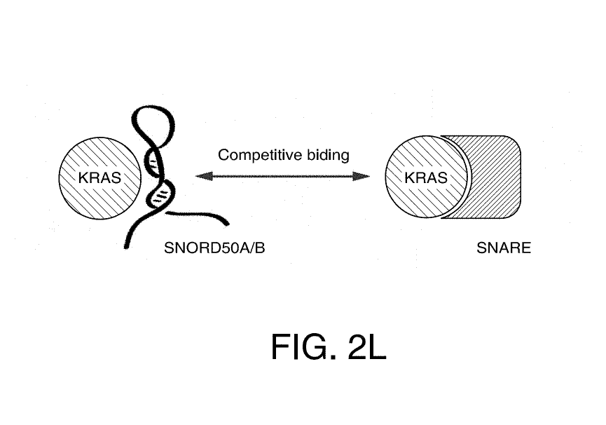

A major caveat of proximity-based proteomics is the inability to distinguish spatial co-localization from direct interaction. To validate that these SNAREs exist in protein complexes with K-RAS, co-immunoprecipitation was performed on endogenous proteins, revealing bi-directional detection of K-RAS-SNAP23 and SNAP23-VAMP3 complexes as well as uni-directional detection of K-RAS-SNAP23, K-RAS-SNAP29, K-RAS-VAMP3, and SNAP23-SNAP29 complexes (FIG. 2a).

Next, K-RAS proximity labeling was repeated with mutations or deletions in Ras functional domains (FIG. 2b). As expected, deletion of either “switch” domain involved in effector binding reduces interaction with Raf1 but did not uniformly alter SNARE interactions (FIG. 2c). Deletion of the hypervariable domain important for membrane localization disrupts interactions both with Raf1 and with SNAREs suggesting that proper membrane association is a common condition for many K-RAS interactions. A previous study on K-RAS-SNORD50A/B interaction identified the positive residues LysS, Lys42, Arg149 and Arg161 as important for SNORD50A/B binding by CLIP.

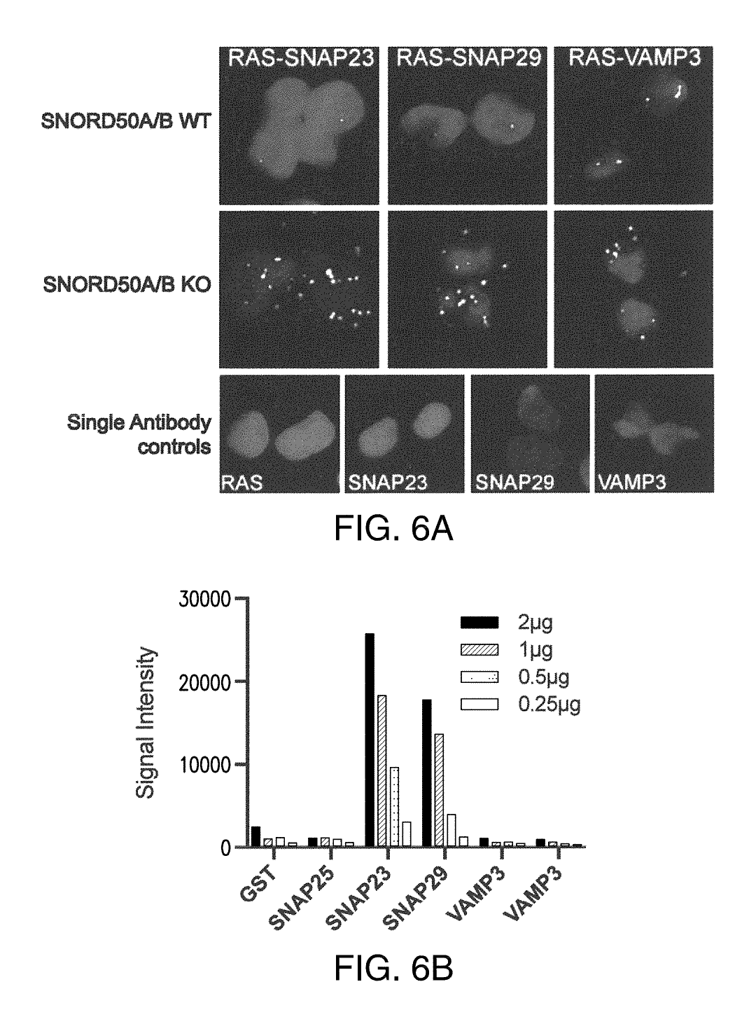

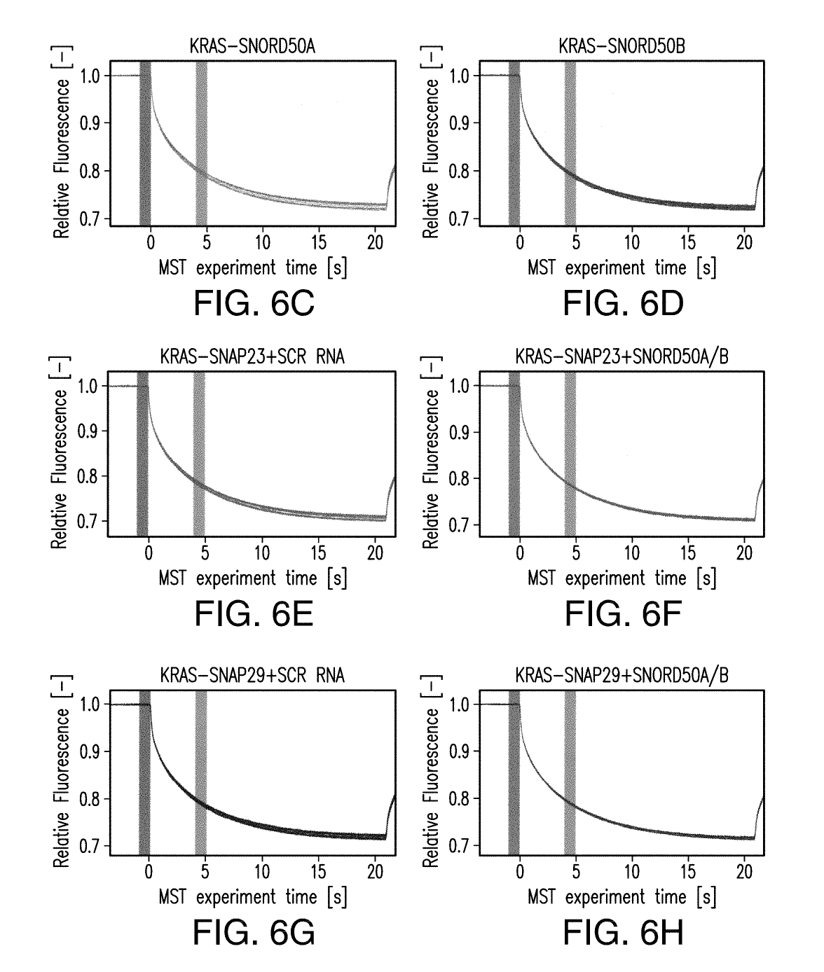

Mutagenesis of these same residues disrupts SNARE interactions without altering Raf1, indicating that SNORD50A/B and SNAREs might share a similar binding surface on K-RAS. The involvement of the same residues in SNORD50A/B and SNARE binding together with the previously mentioned proximal proteomics data strongly suggests that SNORD50A/B inhibit SNARE associations with K-RAS. To test this with an orthogonal approach to proximal proteomics, the relative frequencies of K-RAS-SNARE co-localization in SNORD50A/B WT and KO cells was measured by proximity-based DNA ligation and corroborative evidence that there is an increase in K-RAS-SNARE interaction in the absence of SNORD50A/B was found (FIG. 2d, FIG. 6a).

Next, the ability of each SNARE to directly bind K-RAS without the addition of co-factors was assessed by far western blotting. Of the three SNARE proteins, immobilized SNAP23 and SNAP29 could capture K-RAS from solution (FIG. 2e, FIG. 6b), and a similar competition experiment with RNA in solution reveals that SNORD50A/B, and not a scrambled SNORD50A/B sequence, inhibits binding between K-RAS and SNAP23/SNAP29 (FIG. 2f,g). Binding affinities of direct interactions were then measured by microscale thermophoresis. In this assay, K-RAS binds SNORD50A and SNORD50B with dissociation constants of 140 nM and 122 nM respectively (FIG. 2h,i FIG. 6c,d).

To test the binding affinity of K-RAS to SNARE proteins and the effect of SNORD50A/B on binding affinity, SNORD50A/B and a scrambled SNORD50A/B were co-incubated in MST experiments between K-RAS-SNAP23 and K-RAS-SNAP29. In the presence of scrambled SNORD50A/B, K-RAS bound SNAP23 and SNAP29 with dissociation constants of 177 nM and 178 nM (FIG. 2j,k FIG. 6e-h). In the presence of SNORD50A/B, however, K-RAS-SNAP23 and K-RAS-SNAP29 interactions became undetectable in the same ligand concentration range. Therefore, it was tested whether SNORD50A/B competes with SNAREs for K-RAS binding and whether this competitive binding may in part be responsible for their tumor suppressive action (FIG. 2l).

While in vitro experiments show competitive binding between SNORD50A/B and SNAREs, the functional impact required further inquiry. Next, it was tested whether SNARE binding regulates subcellular trafficking of K-RAS. The Ras isoforms: K-RAS, HRAS and NRAS are each implicated in various human cancers, but K-RAS is an outlier with regard to subcellular trafficking (Rajalingam et al, Castellano et al.). While HRAS and NRAS have removable palmotylation marks that facilitate membrane association, K-RAS has an unalterable, sequence-encoded poly-basic region that mimics lipidation in the same C-terminal domain. For K-RAS to remain enriched at the plasma membrane where it mediates receptor tyrosine kinase signal transduction to downstream signaling pathways, it must be solubilized from vast endomembrane spaces by PDE8 (Chandra et al.).

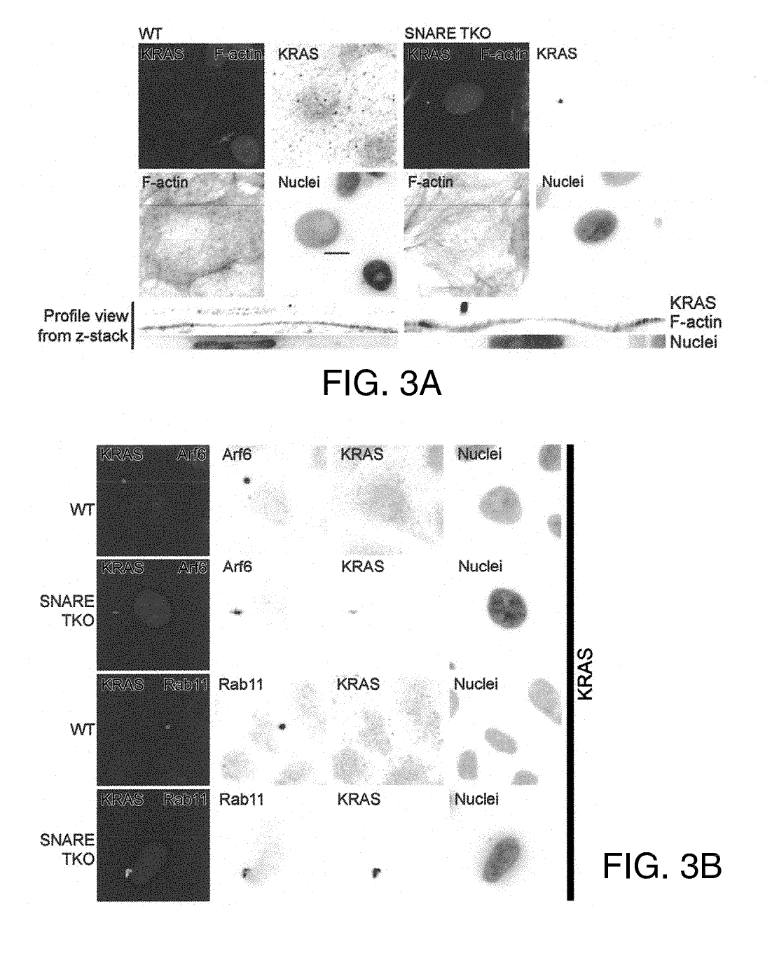

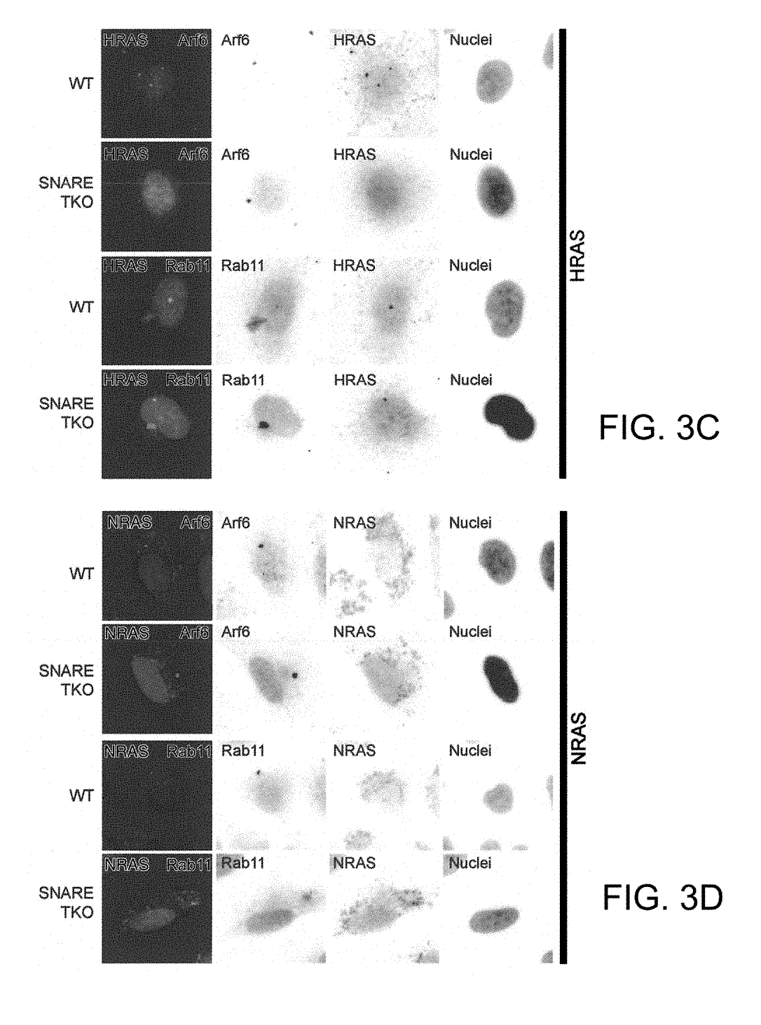

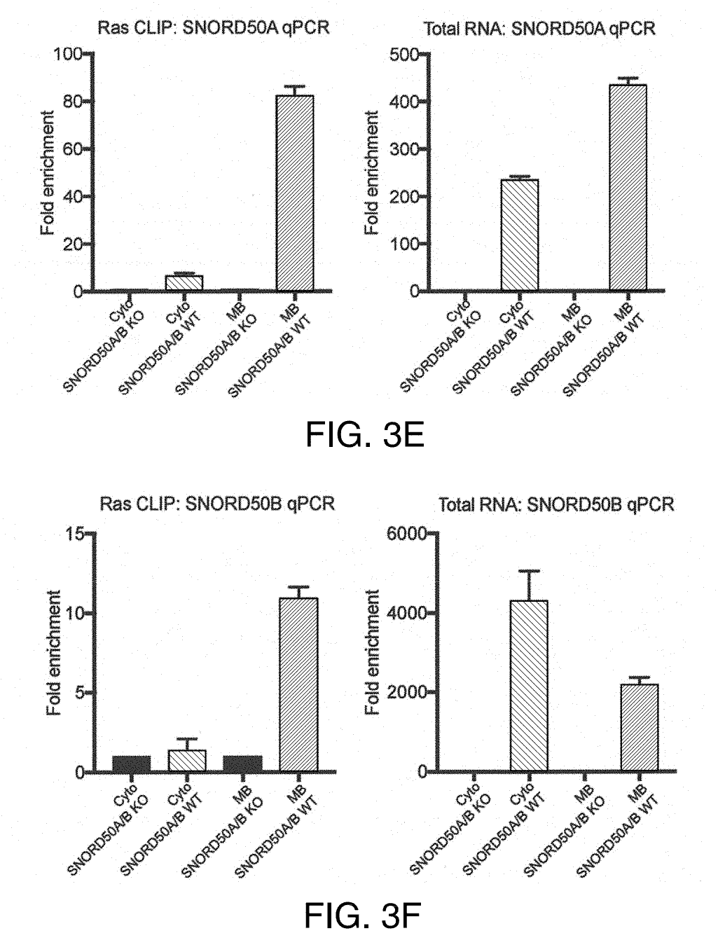

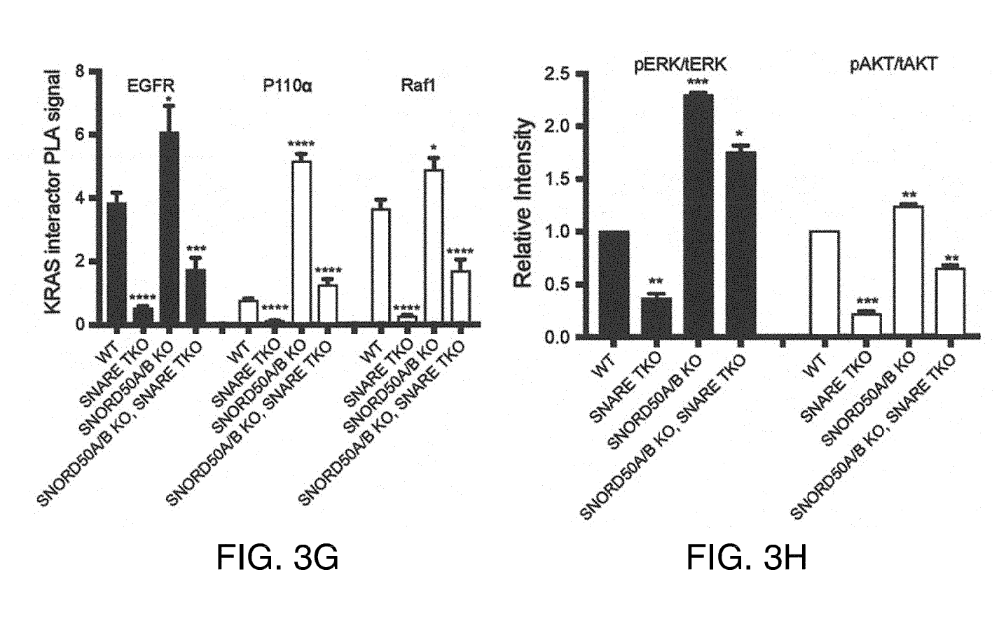

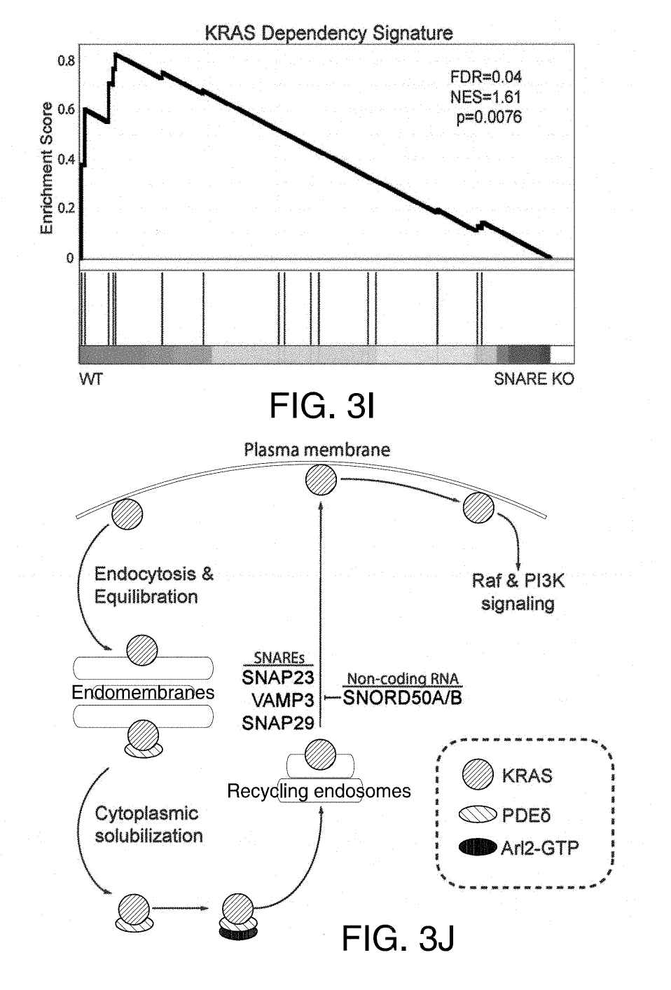

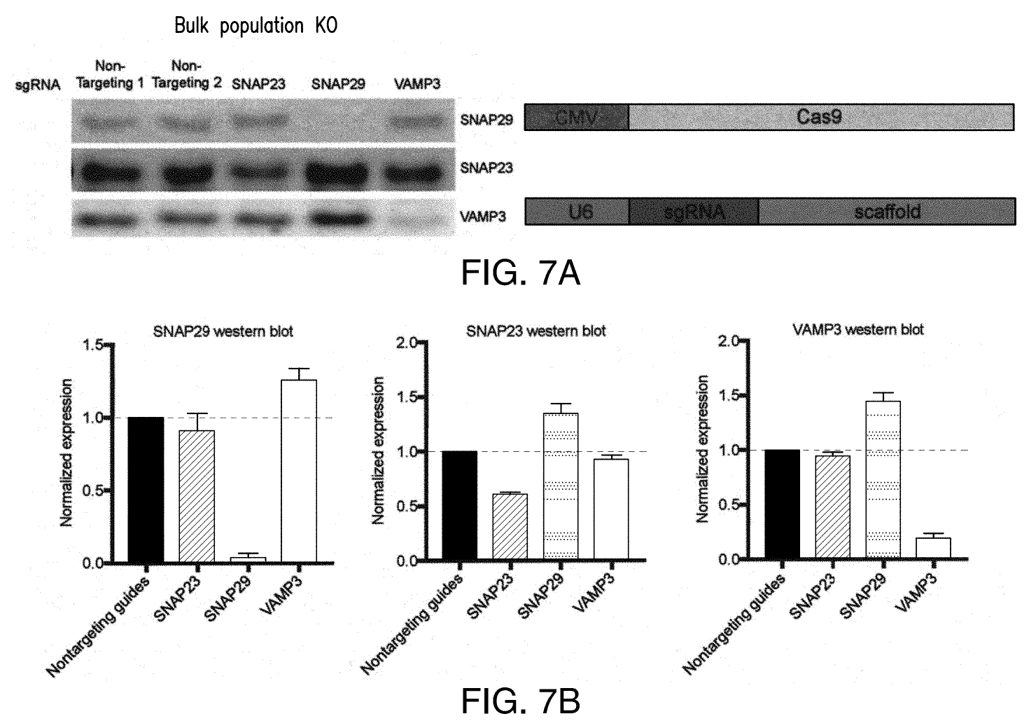

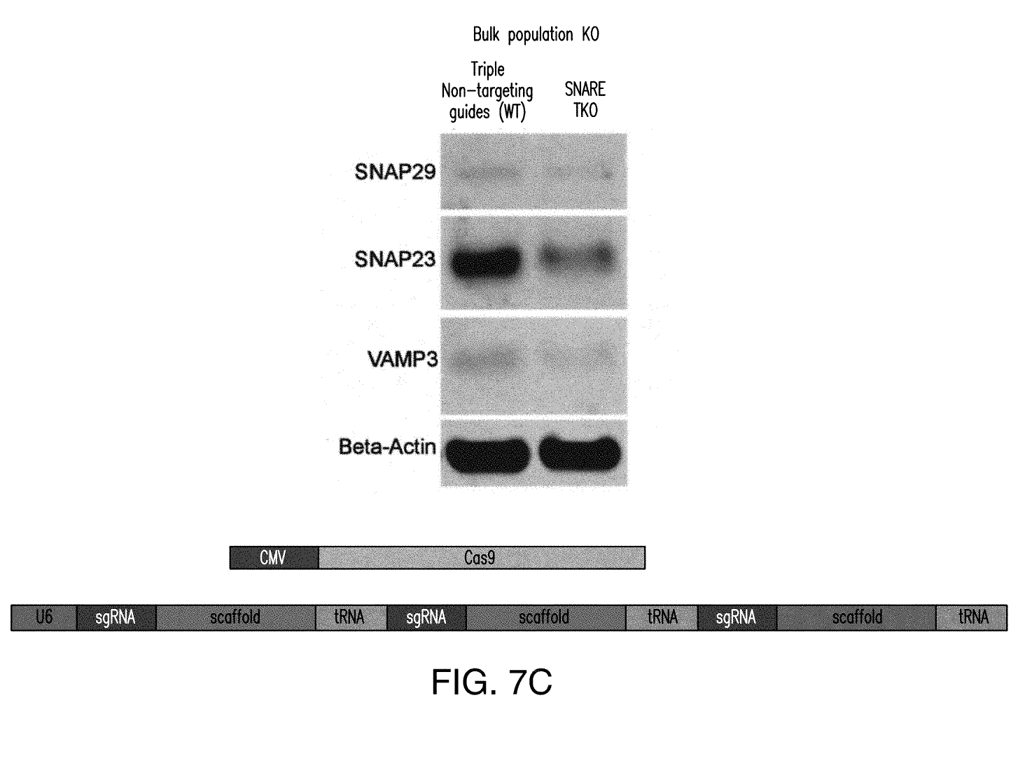

Solubilized K-RAS is then deposited onto recycling endosomes by ARL2 in order to direct its trafficking back to the PM (Schmick et al.). This final step in recycling has been proposed but not clearly demonstrated to involve vesicular transport. High resolution confocal microscopy of K-RAS reveals colocalization with cortical actin at the PM in WT cells (FIG. 3a). In the presence of SNAP29, VAMP3, and SNAP23 triple KO (TKO) (FIG. 7a-c) (Port et al.), however, K-RAS accumulates in a clustered distribution. This cluster of K-RAS co-localized with Arf6 and Rab11 (FIG. 3b), suggesting sequestration into recycling endosomes. Consistent with known differences between trafficking of the Ras isoforms, HRAS and NRAS did not change in subcellular localization upon SNARE TKO (FIG. 3c,d). To check whether SNORD50A/B competitive inhibition is consistent with inhibition of this trafficking step, crosslinking and immunoprecipitation (CLIP) was performed on Ras in cytoplasmic and membrane-bound cellular fractions (FIG. 3e,f).

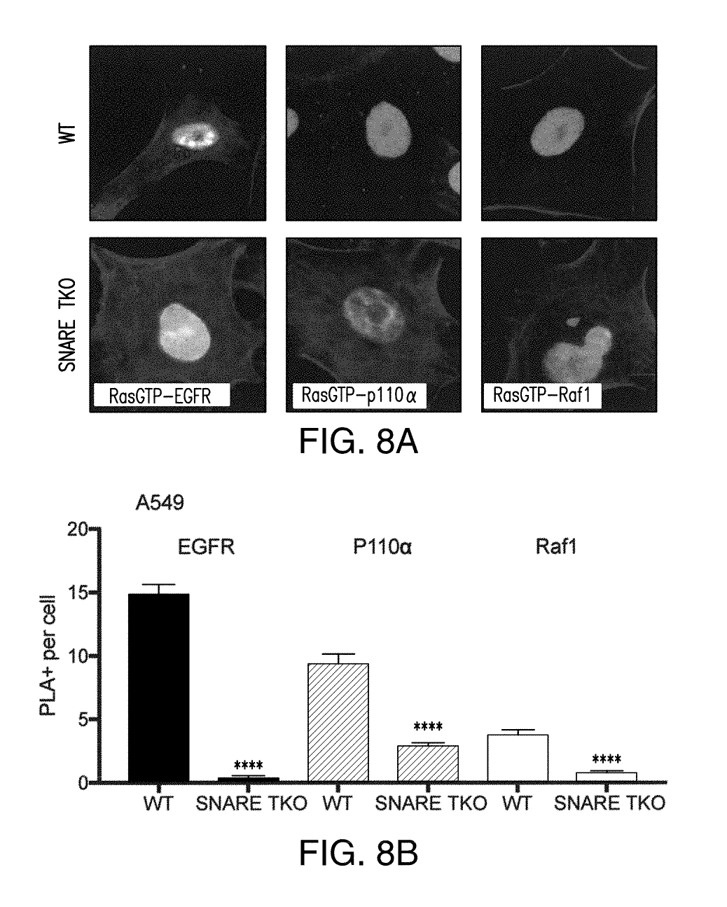

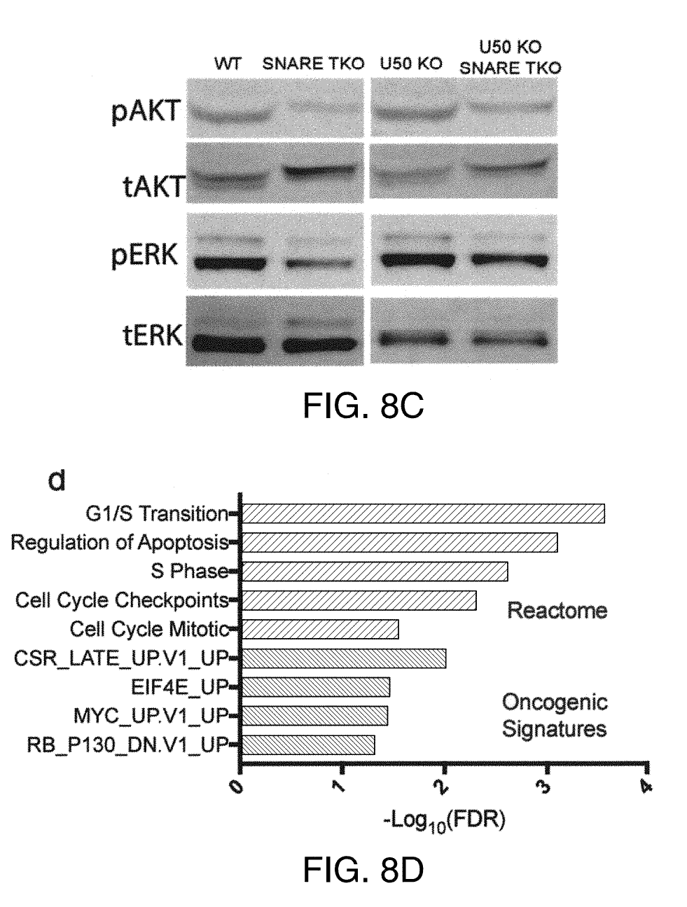

While both SNORD50A and SNORD50B are expressed within two-fold in cytoplasmic vs. membrane fractions, Ras-bound SNORD50A/B is approximately eight-fold more enriched on membrane compartments, thus indicating that K-RAS-SNORD50A/B binding occurs in the appropriate subcellular space to competitively inhibit SNARE binding. Compromised recycling to the plasma membrane should directly affect the ability of K-RAS to associate with membrane receptors and bind effectors. Consistent with this, K-RAS proximity with EGFR and downstream kinases Raf1 and P110a decreased with SNARE TKO and increased with SNORD50A/B KO (FIG. 3g, FIG. 8a,b). This antagonistic regulation could be seen at downstream signaling relays through blotting of pERK and pAKT-SNARE TKO reduced both active ERK and AKT while SNORD50A/B increased active signaling in the two pathways (FIG. 3h, FIG. 8d). The effects of SNARE ablation on K-RAS signaling could also be seen on RNA sequencing. SNARE KOs led to down regulation of a transcriptional signature previously defined as K-RAS-dependent (FIG. 3i, FIG. 8d) (Singh et al.). An important step therefore in sustained K-RAS signaling appears to be SNARE-mediated transport from recycling endosomes to the PM (FIG. 3j).

Although SNARE-mediated transport of K-RAS is important for PM enrichment and sustained signaling, deletion of SNAP29, VAMP3, and SNAP23 likely disrupts additional aspects of vesicular trafficking (Hong et al.). In particular, SNAP23 and VAMP3 have well-established functions in exocytosis (Kean et al.). Although off-target effects of deleting SNARE proteins is an inferior option to direct targeting of the oncogenic protein itself, the resilient nature of K-RAS to therapeutic efforts provides motivation to explore more peripheral dependencies of K-RAS.

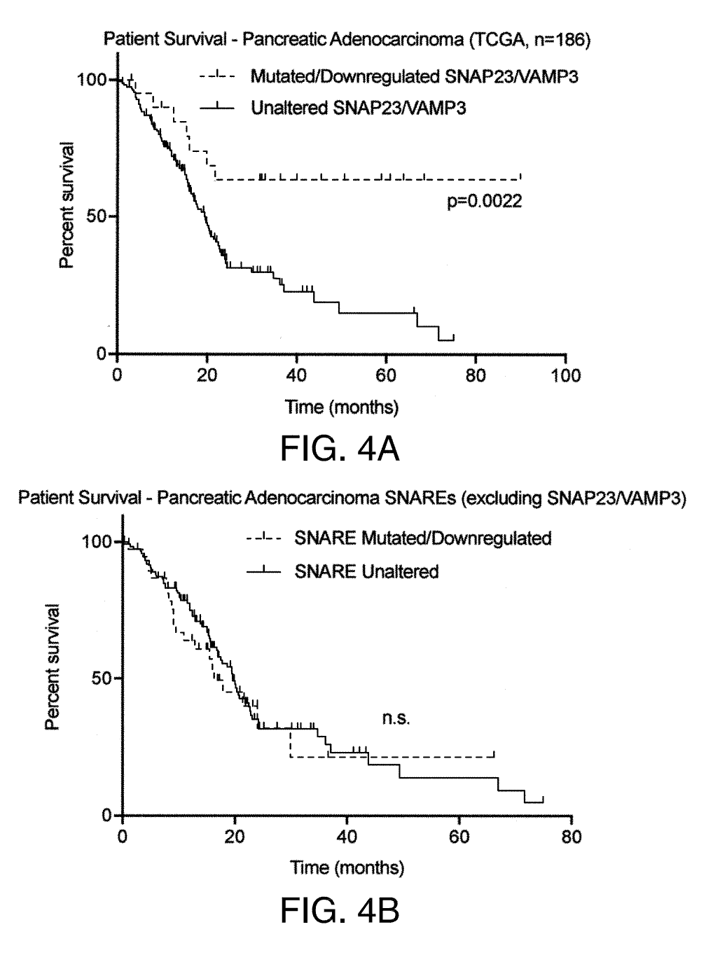

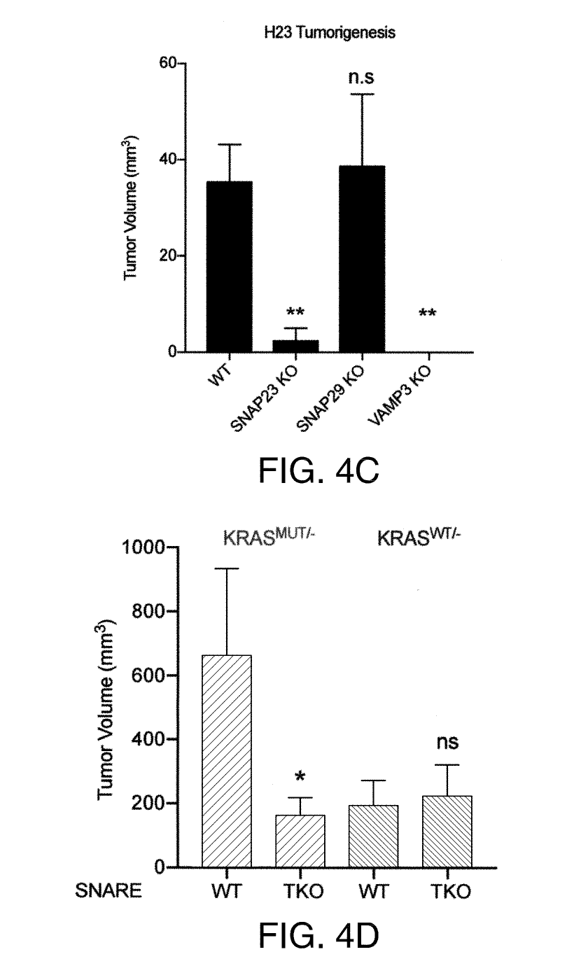

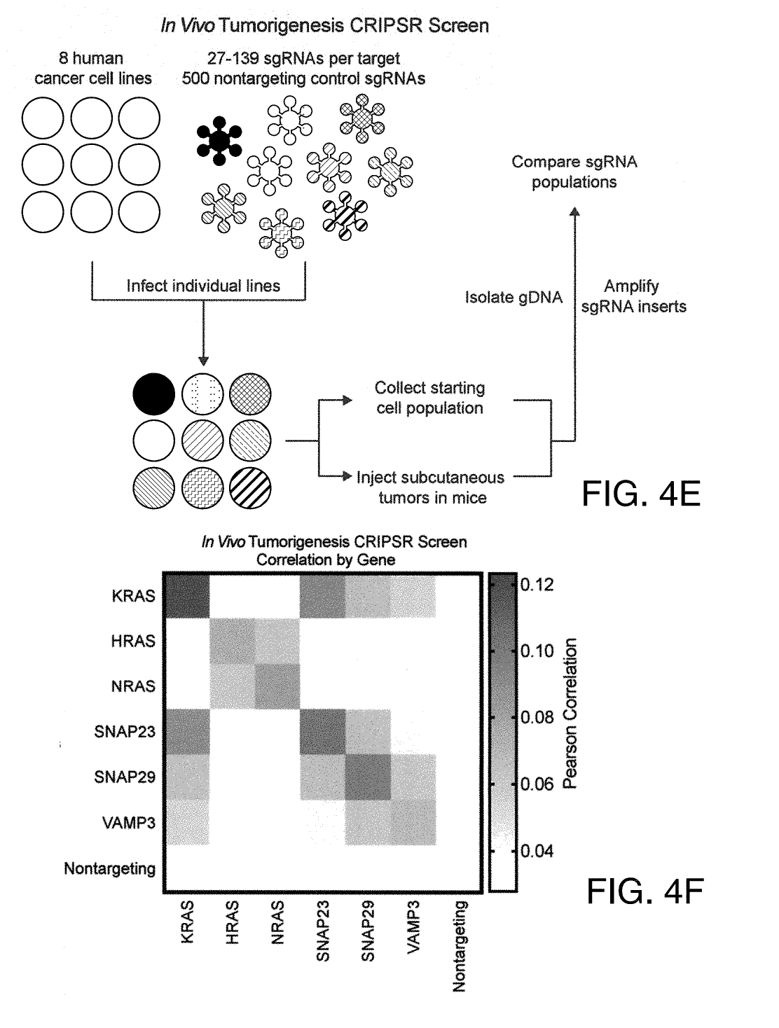

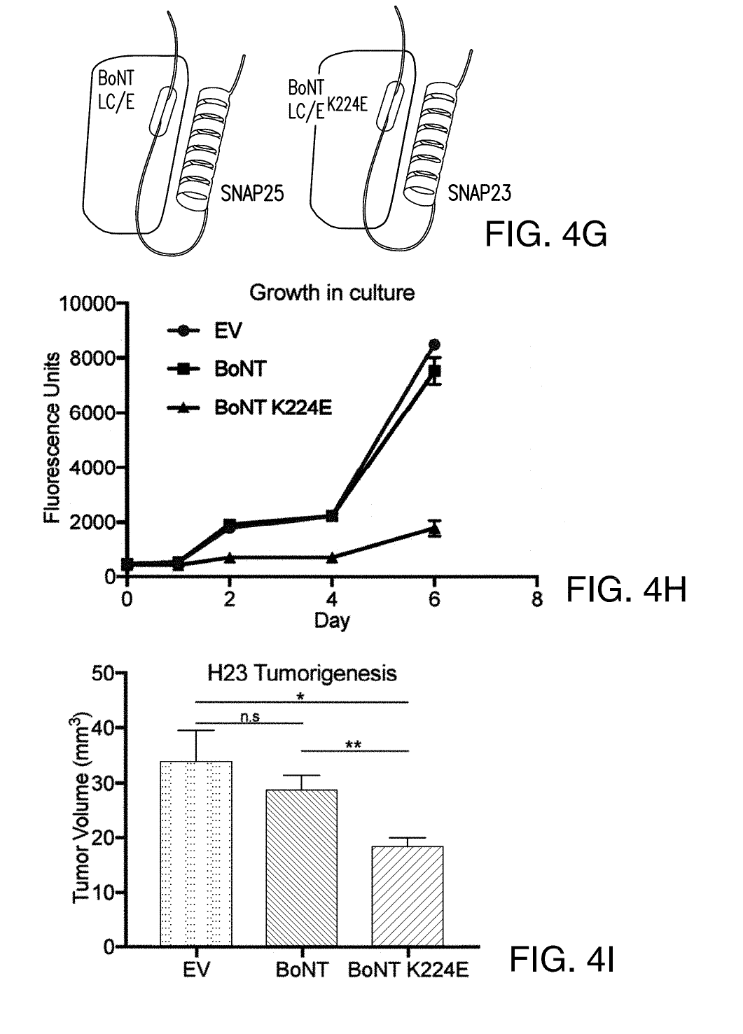

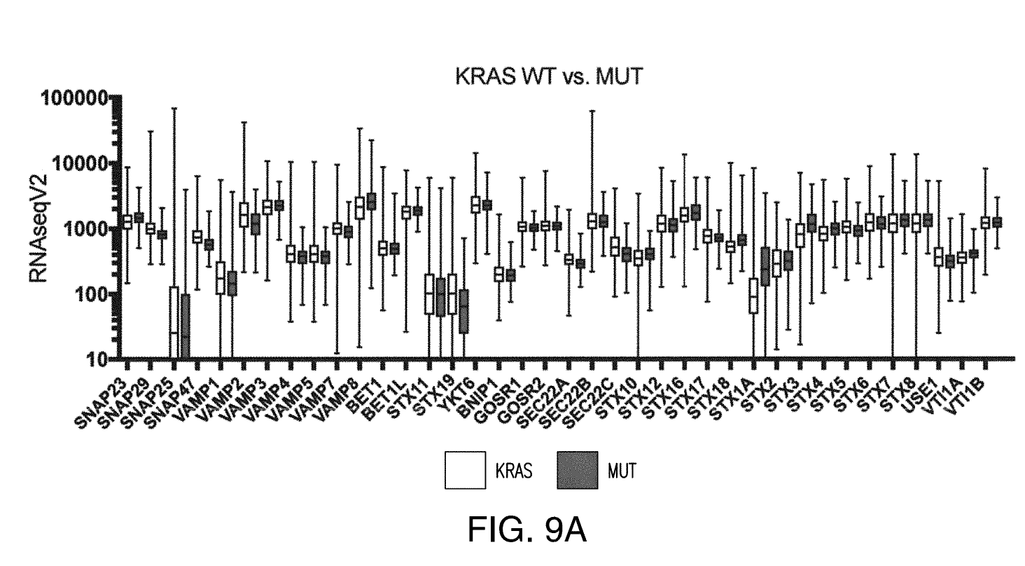

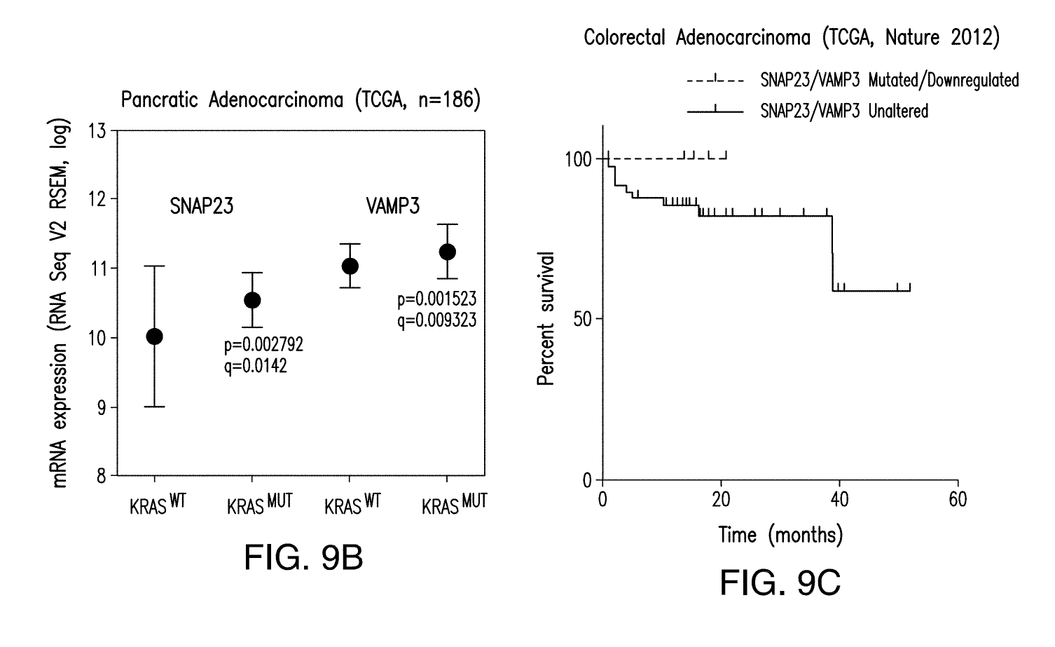

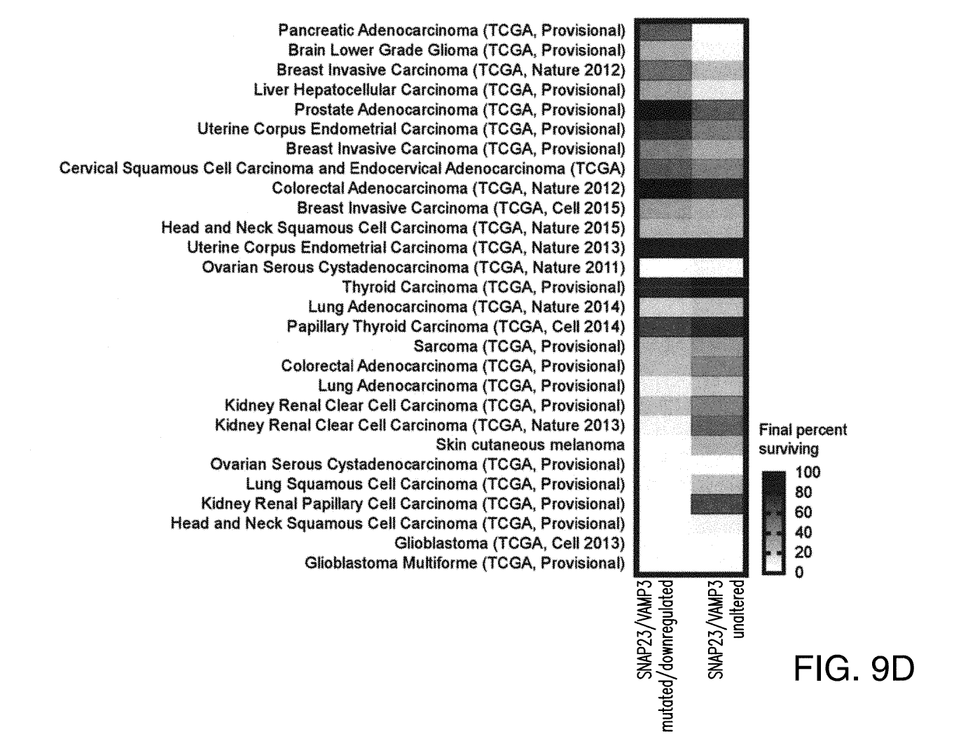

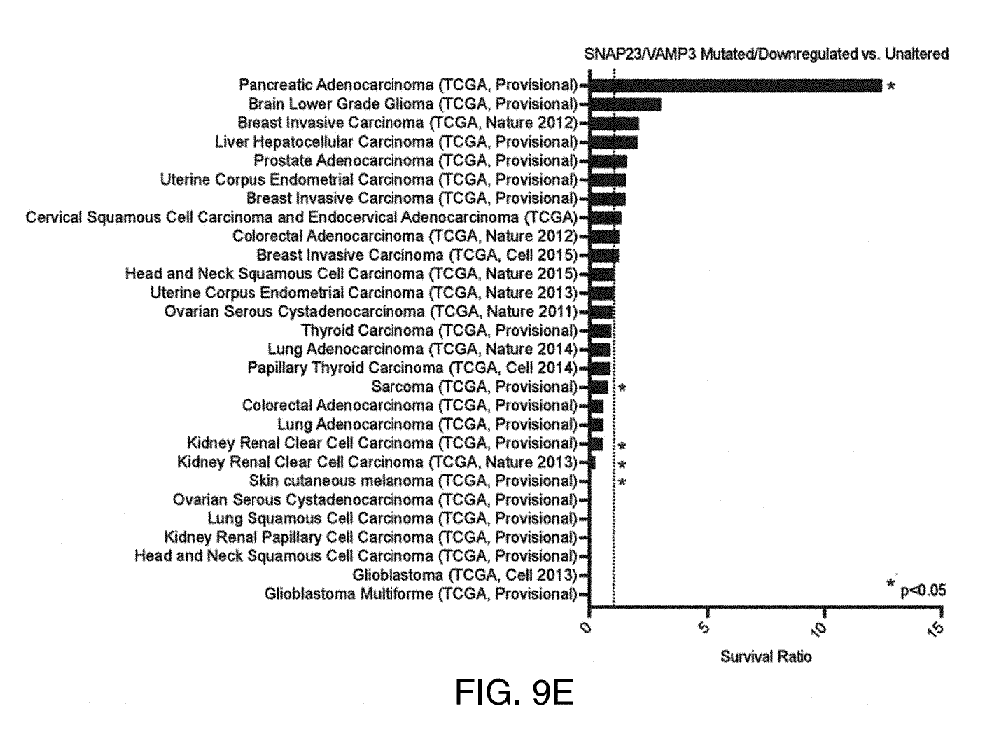

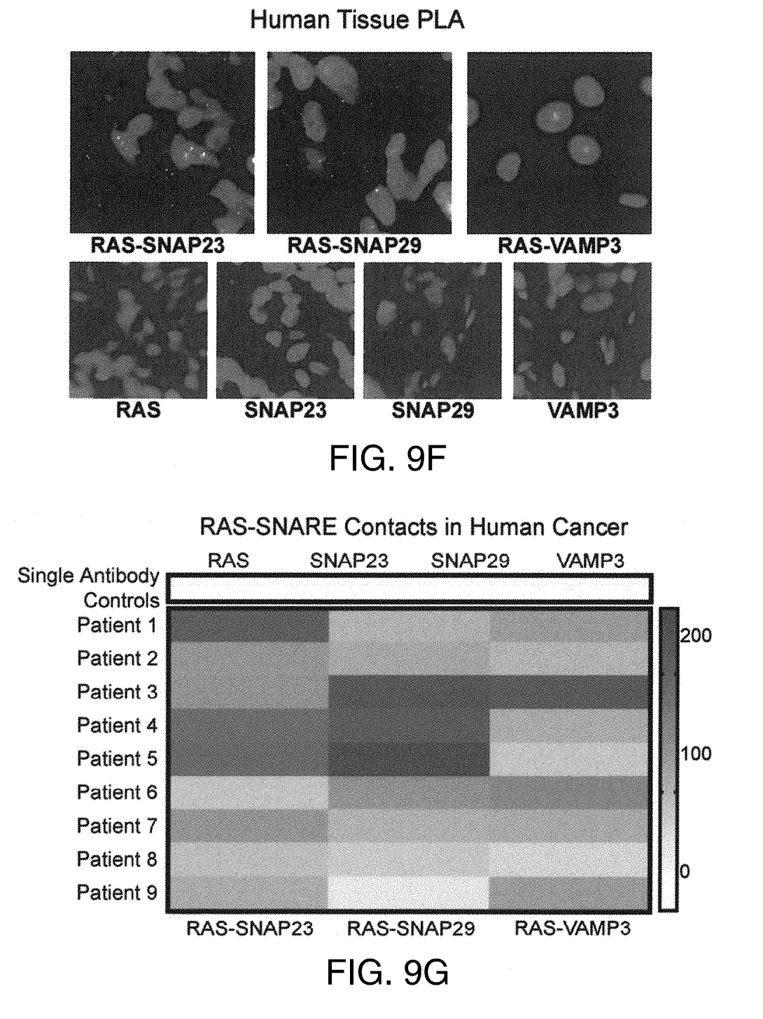

If K-RAS-driven tumors are more dependent on intact SNARE function than normal cells, SNAREs could be targeted within a carefully titrated window. In patient data available in The Cancer Genome Atlas (TCGA) (Cerami et al., Gao et al.), it was found that specifically in pancreatic cancer, which is primarily driven by oncogenic K-RAS mutations, patients with mutated or down regulated SNAP23 or VAMP3 survive longer than their counterparts with unaltered SNAREs (FIG. 4a,b FIG. 9a-d). Interestingly, patients harboring oncogenic K-RAS mutations also maintain higher levels of SNAP23 and VAMP3 expression (FIG. 9e,f). These associations in human cancer along with detectable K-RAS-SNAP29, VAMP3, and SNAP23 interactions by proximity DNA-ligation in sections of human colorectal adenocarcinoma (FIG. 9f,g) led to experiments to test the vulnerability of K-RAS-driven cancers to SNARE KO.

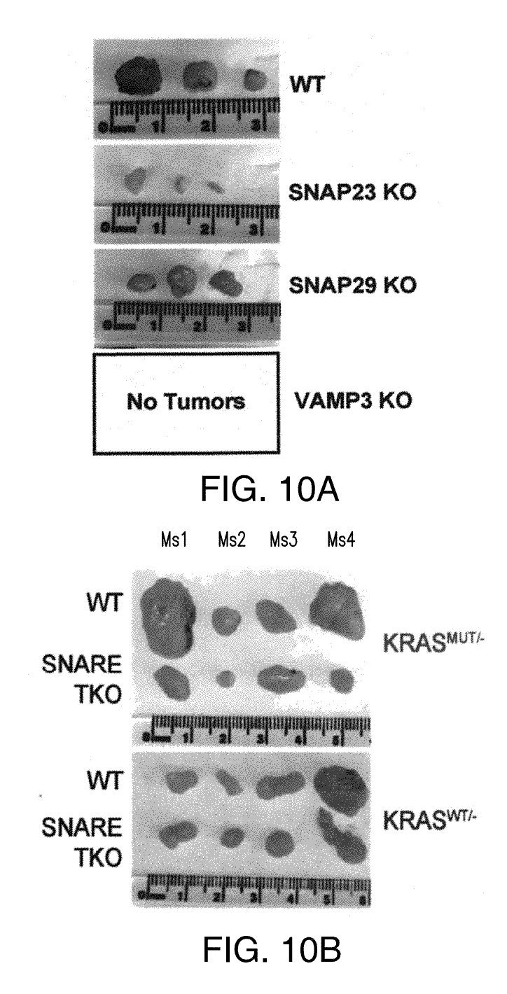

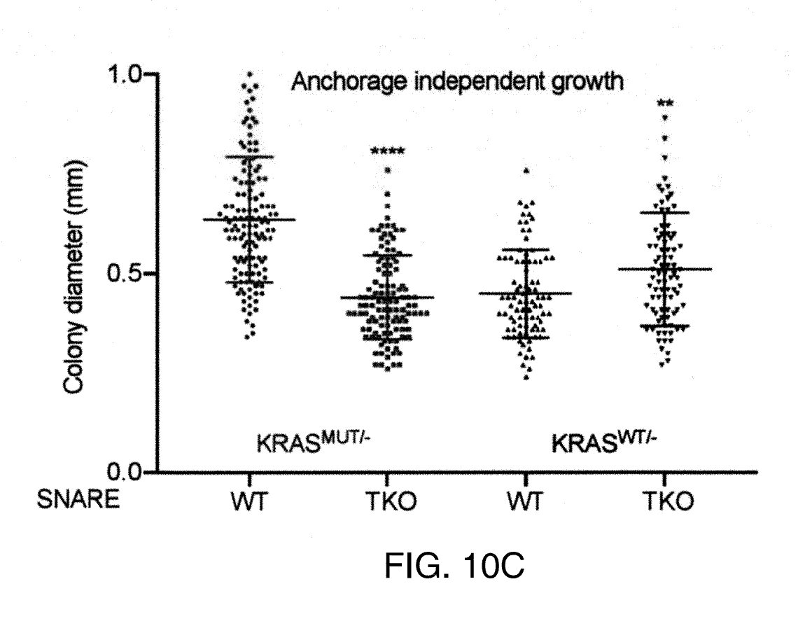

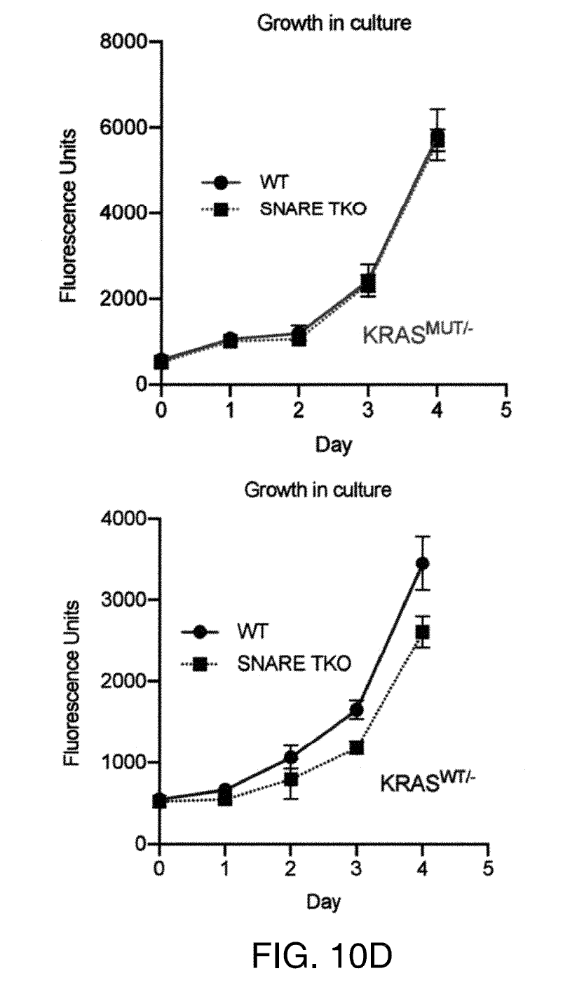

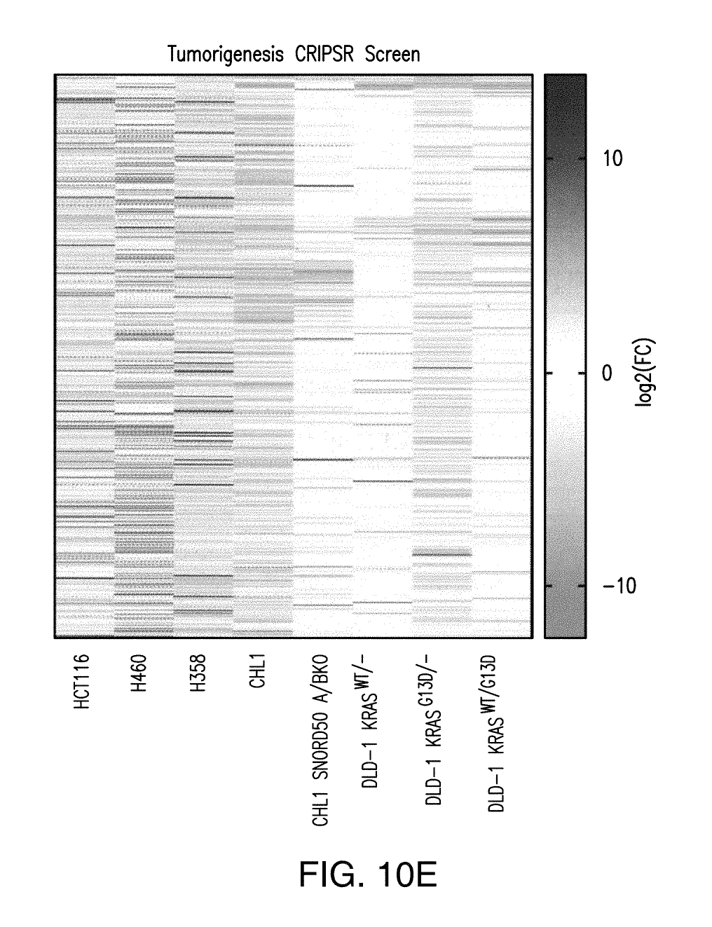

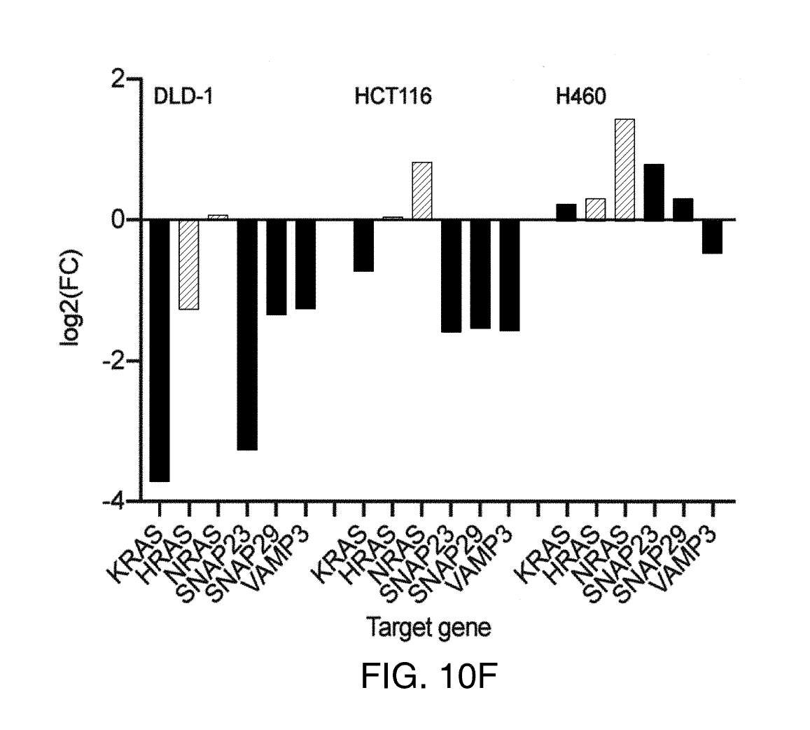

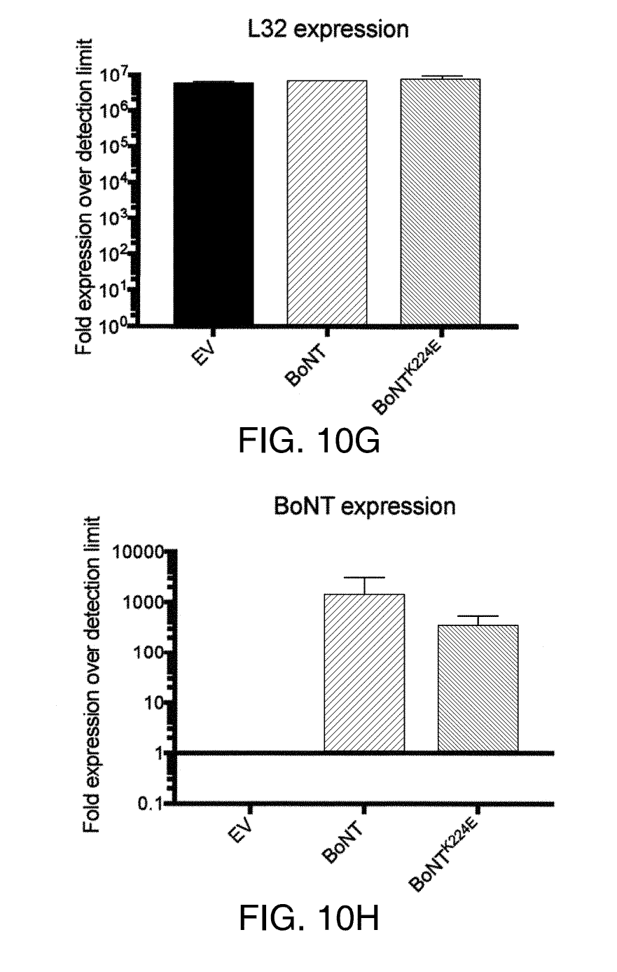

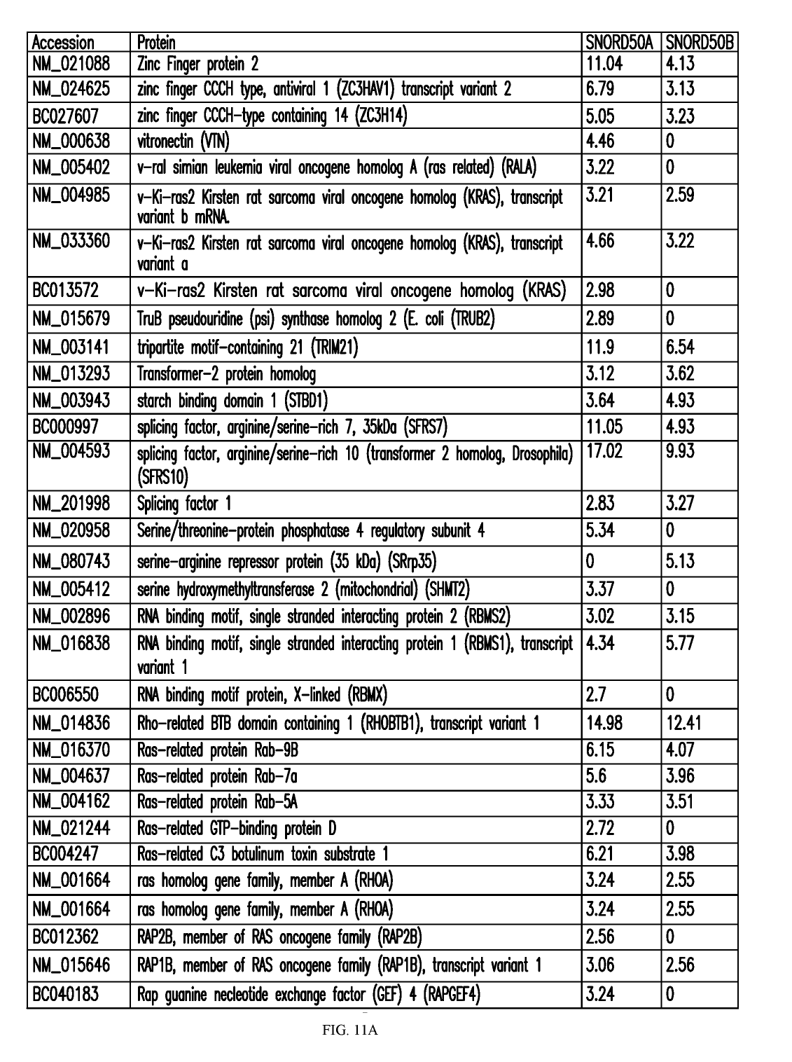

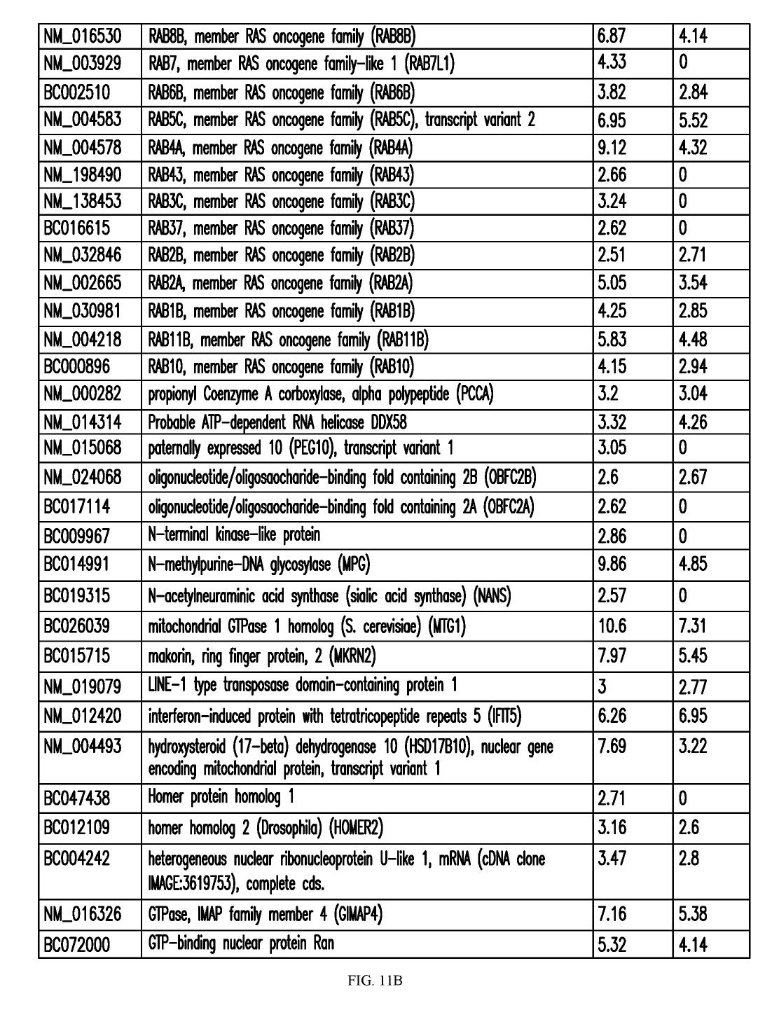

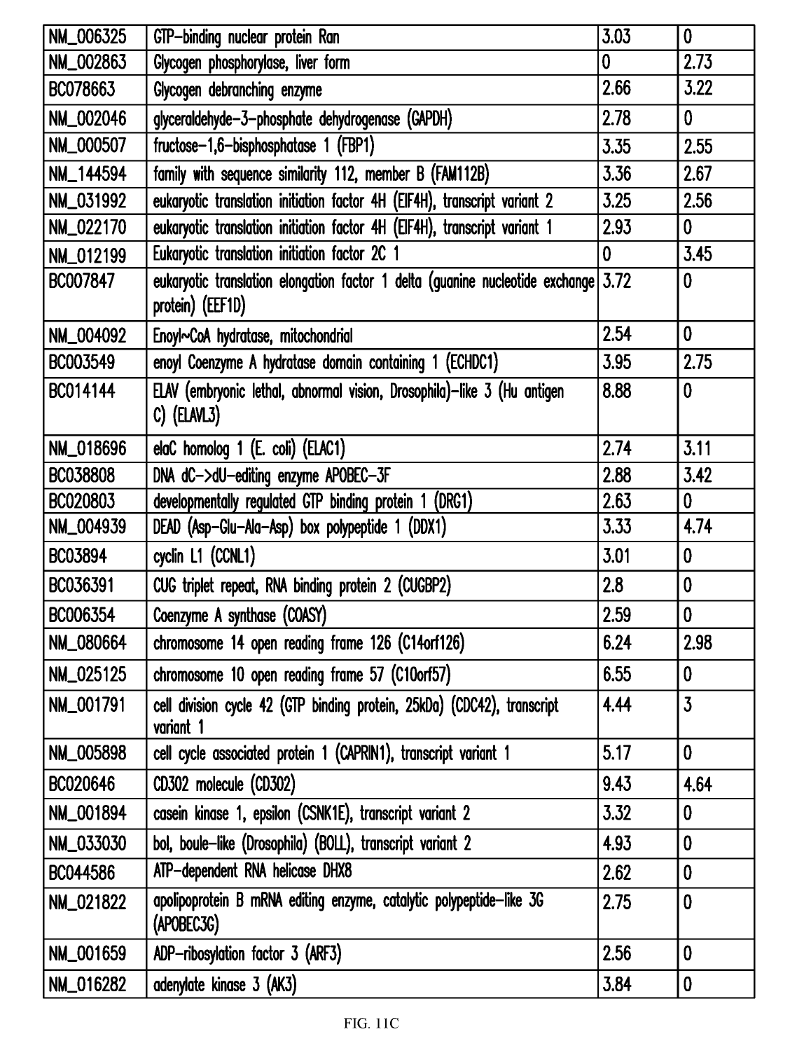

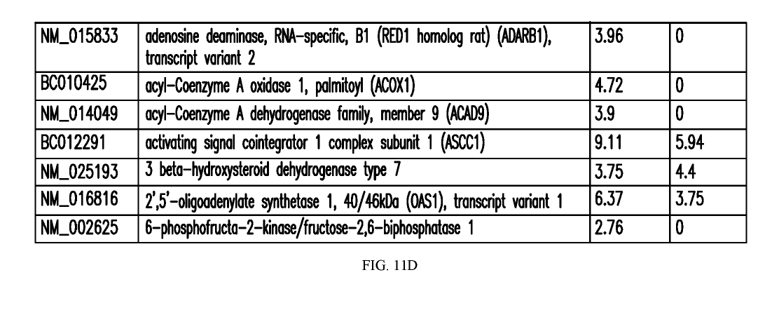

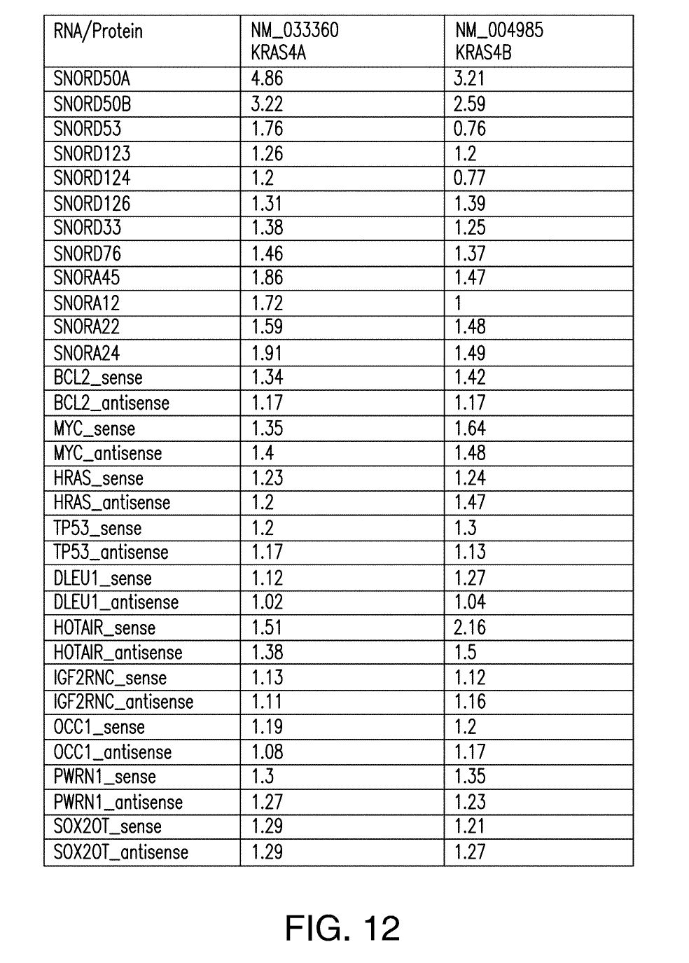

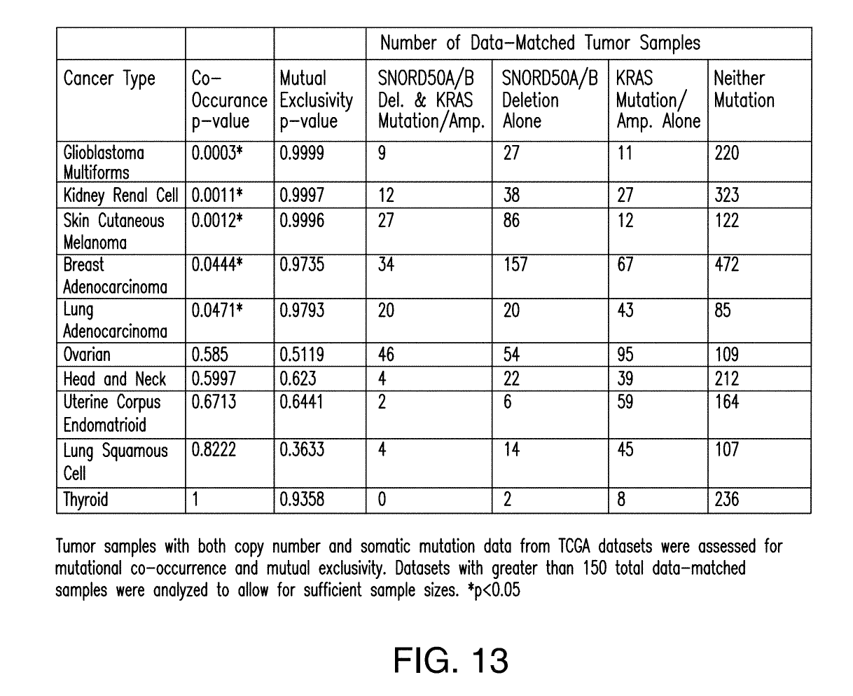

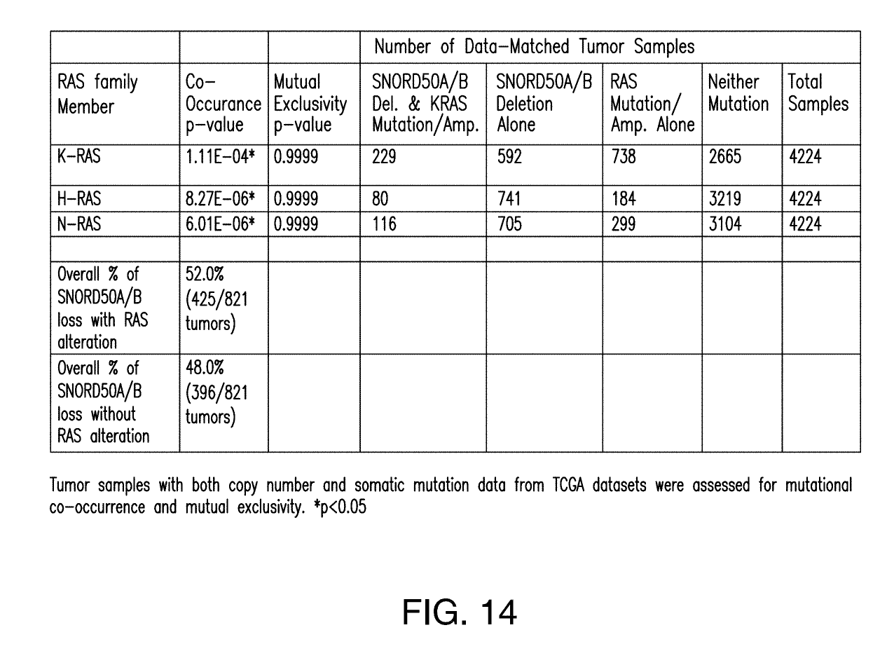

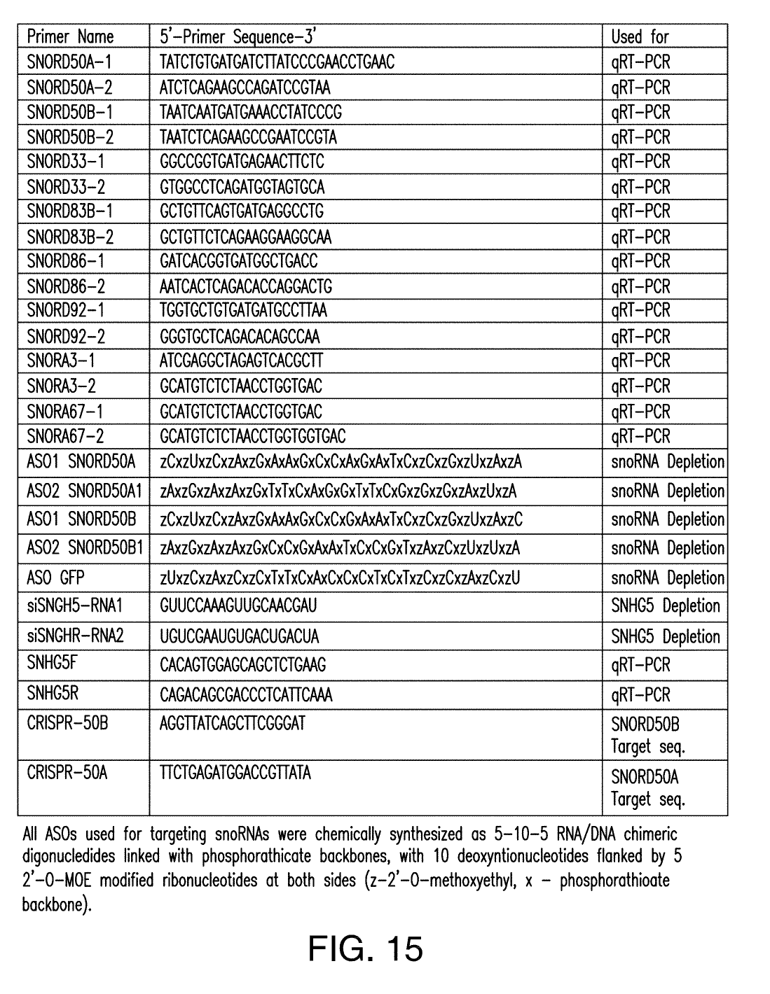

Growth of xenografted subcutaneous tumors reveals dependencies on SNAP23 and VAMP3 (FIG. 4c, FIG. 10a). Similarly designed xenograft experiments were performed in isogenic DLD-1 colon cancer subclones expressing a single WT or oncogenic (MUT) K-RAS allele. SNARE TKO in these tumors reduced the subcutaneous growth of oncogenic K-RAS-driven tumors to basal WT levels (FIG. 4d, FIG. 10b). This specific inhibition of K-RAS mutant cancers in an otherwise identical genetic background could also be seen in anchorage independent growth assays but not in 2D growth (FIG. 10c,d).

Inhibition of SNAREs may therefore be sufficient to derive significant therapeutic benefit in K-RAS-driven cancers without intolerable off-target effects to normal cells. To address the potential non-specific effects of individual guide RNAs and idiosyncrasies of cancer cell lines, an in vivo tumorigensis CRISPR screen with 5 independent and 3 subcloned human cancer cell lines using 1000 guide RNAs was performed to assess the functional link between K-RAS and SNAREs in tumorigenesis (FIG. 4e FIG. 10e,f). Using co-essentiality analysis (Wang et al.), where the phenotypic correlation of genes can be assessed based on depletion or enrichment in numerous cell lines, SNAP23, SNAP29, and VAMP3 correlate with K-RAS but do not correlate with HRAS or NRAS (FIG. 4F).

Finally, vulnerability of K-RAS-driven cancers to SNARE disruption provides rationale for treating K-RAS-driven cancers. Bacterial toxins produced by Clostridium botulinum and Clostridium tetani are capable of cleaving SNARE proteins. In humans, native botulism neurotoxin and tetanus toxin cleave SNAP25 and VAMP2 respectively to disrupt synaptic vesicle fusion. While not immediately applicable, a K224E mutation in the substrate-binding pocket of botulism toxin (BoNT) isoform E, however, renders the toxin active against human SNAP23 (FIG. 4e) (Chen et al.). When expressed in H23s, the catalytic light chain of BoNTK224E dramatically reduces growth in culture and suppresses tumor formation (FIG. 4f,g; and FIG. 10g,h). Given the modular nature of the receptor-binding heavy chain and enzymatic light chain of the toxin, BoNTs can be further engineered for target cell specificity and SNAP23-specificity (Binz et al.). Additionally, BoNT isoform A's success as a therapeutic against disorders of overactive cholinergic nerve terminals suggests that BoNTs can be safely used as therapeutics (Münchau et al.). This approach to targeting K-RAS recycling could be an alternative to or synergistic with drugging the protein directly.

In conclusion, recycling of K-RAS from endosomes back to the plasma membrane is antagonistically regulated by binding to SNARE proteins and non-coding RNAs. These data report that snoRNAs binding protein direct subcellular trafficking and that many small non-coding RNAs annotated as snoRNAs have roles outside of canonical ribosomal RNA modification. Since the discovery of PDEδ-mediated K-RAS solubilization, K-RAS recycling has become a well-appreciated potential cancer target, and efforts have been devoted to targeting PDEδ (Papke et al.).

### Materials And Methods

Cell Culture. Cell lines were grown with 10% FBS and 1% pen-Strep at 37° C., 5% CO2. H23, A549, CHL1 and their respective subcloned lines were cultured in DMEM. H358 and H460 were cultured in RPMI. HCT116, DLD-1 and its respective isogenic lines were cultured in McCoy's 5 A. H23s, A549s, and CHL1s were purchased from ATCC, and DLD-1s were purchased from Horizon. The cell lines were mycoplasma free by the MycoAlert mycoplasma detection kit (Lonza).

Lentivirus Production and Infection. Lentivirus was produced by transfecting 293 Ts with 10 μg pLex lentiviral expression construct, 7.5 μg pCMVA8.91, and 2.5 μg pUC MDG using Transit X2 (Miuras) in a 10 cm plate. Virus was harvested 48 and 72 hrs after transfection and filtered through 0.45 μm filters to remove cell debris and concentrated using Lenti-X concentrator (Clontech). The cell lines were transduced overnight in 5 μg/mL polybrene and selected with 1 μg/mL puromycin or blasticidin when applicable. Cells were selected for a minimum of 2 days for protein expression and 5 days for Cas9-mediated gene editing.

Proximity Protein Labeling/Mass Spectrometry: Sample Preparation. Fusion constructs for vicinal protein labeling were generated in pLex lentiviral expression constructs by expression HA-BirA on the N terminal of K-RAS connected by a 10 amino acid Glycine/serine linker. K-RAS mutants were generated in:

H23, A549, and CHL1 parental cells lines as well as two subcloned lines with homozygous SNORD50A/B knockout that were transduced with pLex-HA-BirA-K-RAS. At ˜80 confluence, media was supplemented with biotin to a final concentration of 50 μM. Cells were harvested 24 hours later (Roux et al., Curr. Protoc. Protein. Sci. 2013) with the addition of a filtration step through a 3K MWCO column prior to MyOne C1 bead binding. For western blotting, 1 mg of total protein input was eluted in 30 μl of elution buffer. For mass spectrometry, an input of ˜10 mgs of total protein was used and half of the eluted product was run on a 4-12% Bis-Tris SDS-PAGE gel and stained with Colloidal Blue (ThermoFischer). A single 1 cm² gel slice per sample was fixed in 50% methanol/10% Acetic acid then stored in 1% acetic acid. Each gel was then digested (Shevchenko et al. Nat. Prot. 2007). Isolated peptides were then reconstituted and injected into a 25 cm C18 reversed phase analytical column in a Waters NanoAcquity run at 450 nL/min from a 4-35% mobile phase (0.1% formic acid). An Orbitrap Elite was set to acquire data selecting and fragmenting 15 precursor ions with the greatest intensity in the ion-trap where the exclusion window was set at 45s and multiple charge states allowed.

Data Analysis. MS/MS data were analyzed using both Preview and Byonic v2.6.49 (ProteinMetrics). Data were first analyzed in Preview to provide recalibration criteria if necessary and then reformatted to .MGF before full analysis with Byonic. Analyses used Uniprot canonical and isoform FASTA files for Human with mutant sequences concatenated as well as common contaminant proteins. Data were searched at 12 ppm mass tolerances for precursors, with 0.4 Da fragment mass tolerances assuming up to two missed cleavages and allowing for fully specific and N-ragged tryptic digestion. These data were validated at a 1% false discovery rate using typical reverse-decoy techniques (Elias Nat. Meth. 2007). The resulting identified peptide spectral matches and assigned proteins were then exported for further analysis using custom tools developed in MatLab (MathWorks) to provide visualization and statistical characterization.

Raw spectral counts were collapsed by gene name and probability scores were calculated for each bait-prey combination using SAINT analysis (crapome.org) using the following parameters: 10,000 iterations, LowMode ON, Normalize ON and the union of MinFold ON and OFF. Available birA controls in crapome.org as well as internal controls were used as background. To identify SNORD50A/B-modulated interactions, probability score change of 0.25 between SNORD50A/B WT and KO datasets and a raw spectral count fold change of >1.5 in at least two of three cell lines were required.

Protein network diagrams were generated in Cytoscape running GeneMANIA to identify protein-protein interactions, pathways, and enrichment scores. Total network connectivity and GO terms for SNORD50A/B-modulated interacions were calculated from the STRING database.

Far Western Blotting. The indicated amounts of recombinant GST(Sigma-Aldrich), SNAP25(abcam), SNAP23(Origene), SNAP29(Origene), VAMP3(LSbiosciences), VAMP3(MyBiosource), RALGDS(Origene), PIK3CG(Origene), or Raf1(Origene) were spotted on a nitrocellulose membrane and allowed to dry for 30 minutes. The membrane was then blocked in 5% milk in TBST and incubated overnight at 4 C with 1 μg K-RAS protein (abcam). For RNA competition far westerns, in vitro transcribed RNA was preincubated with 1 μg K-RAS protein at 4° C. for 1 hr prior to overnight incubation with the membrane. The membrane was then washed in 5% milk in TBST and incubated with horseradish peroxidase (HRP)-conjugated secondary antibody for 1 hr at RT. The membrane was then developed using the SuperSignal West Dura reagent (ThermoFischer). Quantification was done using ImageJ. For loading controls, duplicate membranes were spotted and immediately stained with a Coomassie Stain Kit (Biorad).

In Vitro Transcription. SNORD50A, SNORD50B, and a scrambled sequence based off of SNORD50A/B were transcribed from pCr2.1 TOPO vectors RNAs were then folded in TE buffer by heating to 70 C for 5 minutes and immediately transferring on ice for 15 minutes.

Microscale Thermophoresis. Target proteins were labeled with cysteinereactive dye, NT-647-Red-Melimide (Nanotemper Technologies) in PBS buffer at three molar express to protein solution buffer exchanged into PBS. The protein-dye mixture was incubated at 4° C. for 45 minutes and purified on a desalting column equilibrated with PBS. Binding affinities were analyzed on a Monolith NT.115 instrument (Nanotemper Technologies). Sixteen-point dilutions of indicated ligands were prepared in either PBS (for proteins) or deionized water (for RNA) and mixed 1:1 with labeled target solution so that the final target solution was 20 nM. Reaction mixtures were run on “medium” power and 15% LED power. The dissociation constant Ks is calculated by fitting the binding curve with the quadratic solution for the fraction of fluorescent molecules that formed the complex, calculated from the law of mass action: [AT]=½*(([A0]+[T0]+Kd)−(([A0]+[T0]+Kd)2−4*[A0]*[T0])½) where [A] is the concentration of free fluorescent molecule, [T] the concentration of free titrant and [AT] is the concentration of complexes of [A] and [T], [A0] is the known concentration of the fluorescent molecule and [T0] is the known concentration of added titrant.

Immunofluorescence and Proximity Ligation Assay. Cells were fixed with 4% formaldehyde at RT for 15 minutes then washed twice with PBS. Blocking was done either for 1 hr at RT or overnight at 4° C. in blocking buffer (5% goat serum, 5% horse serum, 0.3% TritonX-100). Primary antibodies were incubated overnight at 4° C. in blocking buffer and secondary antibodies were incubated for 1 hr at RT. Slides were then mounted in Prolong Gold with DAPI (Invitrogen). Primary antibodies used were as follows: anti-Panras C-4 (sc-166691, Santa Cruz Biotechnology), anti-RASGTP (26909, Neweast Bioscienes), anti-K-RAS (12063-1-AP, Proteintech), anti-HRAS (18295-1-AP, Proteintech), anti-NRAS (10724-1-AP, Proteintech), anti-SNAP23 (10825-1-AP, Proteintech), anti-SNAP29 (12704-1-AP, Proteintech), anti-VAMP3 (10702-1-AP, Proteintech), anti-Raf1 C12 (sc-133, Santa Cruz Biotechnology), anti-EGFR 1005 (sc-03-G, Santa Cruz Biotechnology), anti-p110α (4249, Cell Signaling Technologies), anti-RAB11 KT80 (ab95375, Abcam), anti-ARF6 3A-1 (sc-7971, Santa Cruz Biotechnology). Secondary antibodies used were as follows: anti-Rabbit Alexa Fluor 555 (ThermoFischer Scientific), anti-Mouse Alexa Fluor 555 (ThermoFischer Scientific), anti-Rabbit Alexa Fluor 488 (Thermo Fischer Scientific), and anti-Mouse Alexa Fluor 488 (ThermoFischer Scientific).

For PLA, Duolink mouse minus, rabbit plus, and In Situ Orange Reagents (Sigma-Aldrich) were used according to the manufacturer's protocol. When applicable, a cytoskeletal stain using pre-conjugated 488 phalloidin was used (Invitrogen) after polymerase reaction and prior to slide mounting. Images were analyzed using ImageJ.

For patient tumor samples (Asterand Biosciences), prior to blocking, formalin-fixed, paraffin-embedded samples were rehydrated in xylene and sequential dilutions in ethanol followed by Citrate Buffer (pH 6.0) antigen retrieval.

Crosslinking followed by Immunoprecipitation. H23 and a SNORD50A/B KO subclone of the parental line were subjected to 0.3 J/cm2 UV-C crosslinking on ice then subjected to fractionation using the Plasma Membrane Isolation Kit (Abcam) according to manufacturer's instructions. A fraction of each sample was set aside for total RNA isolation and another was resuspended 5 volumes CLIP lysisbuffer (50 mM Tris, pH 7.5, 10% glycerol, 200 mM NaCl, 5 mM EDTA, 0.5% sarkosyl, 0.2% Tween and 0.1% Igepal). The fraction set aside for CLIP was incubated overnight at 4° C. with end over end turning with ProteinG beads (ThermoFischer Scientific) precoupled to an anti-panras C4 antibody. Proteinase K (New England Biosciences) was then added to release RNA. At this time both total RNA as well as CLIP fractions were purified using the miRNeasy Mini Kit (Qiagen). Reverse transcription using the ISCRIPT cDNA synthesis kit (Biorad) with target specific priming for SNORD50A/B was used to generate cDNA, and qPCR was performed using SYBRgreen (ThermoFischer Scientific).

Immunoblot Assays. The following antibodies were used: anti-Panras C-4 (sc-166691, Santa Cruz Biotechnology), anti-RASGTP (26909, Neweast Bioscienes), anti-SNAP23 (10825-1-AP, Proteintech), anti-SNAP29 (12704-1-AP, Proteintech), anti-VAMP3 (10702-1-AP, Proteintech), anti-Phospho-p44/42 MAPK (Erk1/2) (Thr202/Tyr204) (4377, Cell Signaling Technologies), anti-p44/42 Erk1/2 (9107, Cell Signaling Technologies), anti-Phospho-AKT (Ser473) (4060, Cell Signaling Technologies), anti-AKT(pan) (2920, Cell Signaling Technologies).

RNA Sequencing and Gene Set Analysis. RNA sequencing libraries were prepared with Truseqv2 RNA library Prep Kit (Illumina) and sequencing on the HiSeq4000 (Illumina). Alignment was performed with STAR and differential gene expression called with Cuffdiff2. Gene set enrichment analysis was run with 1000 permutations, using gene set permutation and weighted Signal2Noise metrics.

CRISPR-Cas9 mediated Gene Knockout. Cas9 was constitutively expressed using lentiviral infection and blasticidin selection. Single guide were delivered with a standard F+E scaffold. Triple guide vectors were designed with tRNA spacers. For the tumorigenesis library screen, 500 nontargeting sgRNAs and 500 total targeting sgRNAs against K-RAS, HRAS, NRAS, SNAP23, SNAP29, and VAMP3 were constructed. The number of guides chosen was based on the available number of targetable sequences available in the gene exons.

Cell Proliferation and Anchorage Independent Growth Assays. Cell proliferation in culture was measured using the CellTiter Blue Cell Viability Assay (Promega) according to manufacturer instructions. Anchorage independent growth assays were done by plating a base layer of 1:1 ratio 1% agar and McCoy's 5 A (10% FBS) then a top layer 1:1 ratio 0.6% agar and McCoy's 5 A (10% FBS) with cells seeded at the desired density. Colonies were grown at 37° C., 5% CO2 and replenished with media twice per week to avoid dehydration.

Tumorigenesis Studies. Mouse and human tissue were used. Cell lines were first infected with lentiviral vector containing Cas9 and selected in blasticidin for 1 week to obtain a stable population. Cas9-expressing lines were then infected with a lentiviral vector containing single or multiple guide RNA expressing vectors resuspended 2:1 in PBS/matrigel and injected (5e5 for H23 and 5e6 for DLD-1) into the flanks of 8-10 week old female SHO mice (Crl:SHO-PrkdcscidHrhr, Charles River). Tumor volumes were measured with caliper measurements taken by two separate investigators and averaged. Tumor viability in vivo was measured using the IVIS-200 (Xenogen) detecting luciferin metabolism off a stably expressing firefly luciferase. Total luminescence from sites of interest were quantified using LivingImage 4.5 (Caliper LifeSciences). For CRISPR library screens, cell lines were infected to a target of 1000×sgRNA coverage and either injected into the subcutaneous space of SHO mice 2 days post-infection or stored at −80° C. for baseline sgRNA representation in the population. Genomic DNA was isolated from cells or tumors using the DNeasy Blood & Tissue Kit (Qiagen). For tissue obtained from tumors, the tissues was disrupted first using manual dissection and homogenization, and the proteinase K digestion step was extended to overnight. The other procedures were done according to the manufacturer's specifications. Guide cassettes were amplified by PCR using PrimeSTAR Max DNA Polymerase Premix (Takara) in two steps (see PCR primers below), and reactions were cleaned up with AMPure XP beads (Beckman Coulter). Library quality control and quantification were done by BioAnalyzer (Agilent Genomics). Libraries were then pooled and sequenced on a Miseq platform (Illumina) and mapped back to the guide RNA pool. Imperfect matches were discarded and quantification was performed using R.

Library PCR1/2 F:
(SEQ ID NO: 1)
AATGATACGGCGACCACCGAGATCTACACTCTTTCCCTACACGACGCTCT
TCCGATCTTGTGGAAAGGACGAAACACC
Library PCR1 R:
(SEQ ID NO: 2)
GTGACTGGAGTTCAGACGTGTGCTCTTCCGATCGTAATACGGTTATCCAC
GCGG
Library PCR2 R:
(SEQ ID NO: 3)
CAAGCAGAAGACGGCATACGAGATNNNNNNNNGTGACTGGAGTTCAGACG
TG

TCGA Patient Survival and Gene Expression Analysis. Patient data from TCGA including mutations, expression, and clinical outcomes were downloaded through cBioportal. Statistical tests regarding gene expression were down in cBioportal while survival analysis was done in PRISM.

Statistics. Error bars represent standard error of the mean. *p<0.05, **p<0.01, ***p<0.001, ****p<0.0001. Unless otherwise noted, a t-test was used to determine statistical significance. In cases where data was not expected to fall in a normal distribution, Welch's correction was applied.

The following primers were used for quantitative PCR:

SNORD50A:
(SEQ ID NO: 4)
atctcagaagccagatccg,
(SEQ ID NO: 5)
tatctgtgatgatcttatcccgaac;
SNORD5OB:
(SEQ ID NO: 6)
atctcagaagccgaatccg,
(SEQ ID NO: 7)
taatcaatgatgaaacctatcccgaag;
Botulism neurotoxin E:
(SEQ ID NO: 8)
cgacaggacaatattgtatattaaacct,
(SEQ ID NO: 9)
gtttttcaagctcgtcggag

### REFERENCES

Siprashvili, Z. et al. The noncoding RNAs SNORD50A and SNORD50B bind K-Ras and are recurrently deleted in human cancer. Nat. Genet. 48, 53-8 (2016).
Williams, G. T. & Farzaneh, F. Are snoRNAs and snoRNA host genes new players in cancer? Nat. Rev. Cancer 12, 84-8 (2012).
Stephen, A. G., Esposito, D., Bagni, R. K. & McCormick, F. Dragging ras back in the ring. Cancer cell 25, 272-281 (2014).
Prior, I. A., Lewis, P. D. & Mattos, C. A comprehensive survey of Ras mutations in cancer. Cancer Res. 72, 2457-67 (2012).
Pylayeva-Gupta, Y, Grabocka, E & Bar-Sagi, D. RAS oncogenes: weaving a tumorigenic web. Nature Reviews Cancer (2011).
Roux, K. J., Kim, D. I., Raida, M. & Burke, B. A promiscuous biotin ligase fusion protein identifies proximal and interacting proteins in mammalian cells. J. Cell Biol. 196, 801-10 (2012).
Szklarczyk, D. et al. STRING v10: protein-protein interaction networks, integrated over the tree of life. Nucleic Acids Res. 43, D447-52 (2015).
Rajalingam, K, Schreck, R, Rapp, UR & Albert, S. Ras oncogenes and their downstream targets. Biochimica Et Biophysica (2007).
Castellano, E & Santos, E. Functional specificity of Ras isoforms so similar but so different. Genes & cancer (2011).
Papke, B. & Der, C. J. Drugging RAS: Know the enemy. Science 355, 1158-1163 (2017).
Chandra, A. et al. The GDI-like solubilizing factor PDEδ sustains the spatial organization and signalling of Ras family proteins. Nat. Cell Biol. 14, 148-58 (2011).
Schmick, M, Vartak, N, Papke, B & Kovacevic, M. K-ras localizes to the plasma membrane by spatial cycles of solubilization, trapping and vesicular transport. Cell (2014).
Port, F. & Bullock, S. L. Augmenting CRISPR applications in Drosophila with tRNA-flanked sgRNAs. Nat. Methods 13, 852-4 (2016).
Jensen, K. B. & Darnell, R. B. CLIP: crosslinking and immunoprecipitation of in vivo RNA targets of RNA-binding proteins. Methods Mol. Biol. 488, 85-98 (2008).
Singh, A. et al. A gene expression signature associated with ‘K-Ras addiction’ reveals regulators of EMT and tumor cell survival. Cancer Cell 15, 489-500 (2009).
Cerami, E. et al. The cBio cancer genomics portal: an open platform for exploring multidimensional cancer genomics data. Cancer Discov 2, 401-4 (2012).
Gao, J. et al. Integrative analysis of complex cancer genomics and clinical profiles using the cBioPortal. Sci Signal 6, p 11 (2013).
Hong, W. SNAREs and traffic. Biochimica et Biophysica Acta (BBA)-Molecular Cell (2005).
Wang, T. et al. Gene Essentiality Profiling Reveals Gene Networks and Synthetic Lethal Interactions with Oncogenic Ras. Cell 168, 890-903.e15 (2017).
Chen, S. & Barbieri, J. T. Engineering botulinum neurotoxin to extend therapeutic intervention. Proc. Natl. Acad. Sci. U.S.A. 106, 9180-4 (2009).
Kean, M. J. et al. VAMP3, syntaxin-13 and SNAP23 are involved in secretion of matrix metalloproteinases, degradation of the extracellular matrix and cell invasion. J. Cell. Sci. 122, 4089-98 (2009).
Binz, T., Sikorra, S. & Mahrhold, S. Clostridial neurotoxins: mechanism of SNARE cleavage and outlook on potential substrate specificity reengineering. Toxins (Basel) 2, 665-82 (2010).
Münchau, A. & Bhatia, K. P. Uses of botulinum toxin injection in medicine today. BMJ 320, 161-5 (2000).

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of disrupting RAS-driven cancer growth in a mammalian cell, said method comprising administering to said cell an amount of a toxin derived from an engineered botulism toxin mutant effective in cleaving a SNARE protein that is essential for RAS signaling, thereby disrupting RAS-driven cancer growth.

2. The method of claim 1, wherein the SNARE protein is SNAP-23.

3. A method of treating a patient affected with oncogenic Ras-driven cancer, the method comprising administering to patient a therapeutically effective amount of a botulism toxin (BoNT) isoform E mutant or biologically active variant thereof, wherein BoNT isoform E cleaves SNAP23.

4. The method of claim 3, wherein the patient is a human patient.

5. The method of claim 3, wherein the cancer is lung cancer, colon cancer, pancreatic cancer, colorectal cancer, cervical cancer, head and neck squamous cell carcinoma, thyroid cancer, melanoma, bladder cancer, liver cancer, or kidney cancer.

6. The method of claim 3, wherein the BoNT isoform E mutant is adminstered parentally or intravenously.

7. The method of claim 6, wherein the parental administration is intravenous, subcutaneous, intramuscular or direct injection.

8. A method of reducing a tumor in a subject, wherein the tumor has a constitutively activating mutation of Ras, the method comprising administering to the subject a therapeutically effective amount of botulism toxin (BoNT) isoform E mutant, wherein the BoNT isoform E mutant cleaves SNAP23.

9. The method of claim 8, wherein the subject is a human.

10. The method of claim 8, wherein BoNT isoform E mutant is administered in combination with one or more chemotherapeutic or anti-cancer therapeutics or in combination with radiotherapy or immunotherapy.

11. The method of claim 8, wherein the BoNT isoform E mutant comprises a K224E mutation.

12. The method of claim 8, wherein the tumor is in the subject's pancreas, lung, colon, rectum, thyroid, bladder, liver, kidney, skin, cervix, head or neck.

13. A method of inhibiting or reducing K-RAS activity, the method comprising contacting a cell or tissue with a therapeutically effective amount of an engineered bacterial SNARE-cleaving toxin.

14. The method of claim 13, wherein the SNARE-cleaving toxin is a toxin from an engineered Botulism toxin E mutant.

15. The method of claim 13, wherein the engineered Botulism toxin E mutant comprises a K224E mutation.

16. The method of claim 13, wherein the SNARE protein is SNAP-23.

17. A method of treating a subject with cancer, the method comprising: (a) identifying a subject in need of treatment; and (b) administering to the subject a therapeutically effective amount of a toxin from an engineered Botulism toxin E mutant.

18. The method of claim 17, wherein the subject is a human.

19. The method of claim 17, wherein the subject has been diagnosed with cancer prior to the administering step.

20. The method of claim 17, wherein the cancer is a primary or secondary tumor.

21. The method of claim 20, wherein the primary or secondary tumor is within the subject's pancreas, lung, colon, rectum, thyroid, bladder, liver, kidney, skin, cervix, head or neck.

22. The method of claim 17, wherein the cancer is lung cancer, colon cancer, pancreatic cancer, colorectal cancer, cervical cancer, head and neck squamous cell carcinoma, thyroid cancer, melanoma, bladder cancer, liver cancer, or kidney cancer.

23. The method of claim 17, wherein the toxin from an engineered Botulism toxin E mutant is administered orally or parentally.

24. The method of claim 23, wherein the parental administration is intravenous, subcutaneous, intramuscular or direct injection.


SEQUENCE LISTING (9 sequences)

SEQ ID NO: 1 - DNA, 78 bp, Artificial Sequence
  Synthetic construct
aatgatacggcgaccaccgagatctacactctttccctacacgacgctcttccgatcttgtggaaaggacgaaacacc

SEQ ID NO: 2 - DNA, 54 bp, Artificial Sequence
  Synthetic construct
gtgactggagttcagacgtgtgctcttccgatcgtaatacggttatccacgcgg

SEQ ID NO: 3 - DNA, 52 bp, Artificial Sequence
  Synthetic construct
  misc_feature (25)..(32): n is a, c, g, or t
caagcagaagacggcatacgagatnnnnnnnngtgactggagttcagacgtg

SEQ ID NO: 4 - DNA, 19 bp, Artificial Sequence
  Synthetic construct
atctcagaagccagatccg

SEQ ID NO: 5 - protein, 25 aa, Artificial Sequence
  Synthetic construct
TATCTGTGATGATCTTATCCCGAAC

SEQ ID NO: 6 - DNA, 19 bp, Artificial Sequence
  Synthetic construct
atctcagaagccgaatccg

SEQ ID NO: 7 - DNA, 27 bp, Artificial Sequence
  Synthetic construct
taatcaatgatgaaacctatcccgaag

SEQ ID NO: 8 - DNA, 28 bp, Artificial Sequence
  Synthetic construct
cgacaggacaatattgtatattaaacct

SEQ ID NO: 9 - DNA, 20 bp, Artificial Sequence
  Synthetic construct
gtttttcaagctcgtcggag